(19) United States
(12) Patent Application Publication
(10) Pub. No.: US 2026/0321364 A1
(43) Pub. Date: Oct. 15, 2026

(54) EXPLAINABLE MACHINE LEARNING SYSTEMS AND METHODS FOR DATA DISCOVERY AND INSIGHT GENERATION

(71) Applicant: MINED XAI LLC, Dayton, OH (US)

(72) Inventors: Ryan Kramer, Bellbrook, OH (US); Kenneth Howe, Dayton, OH (US); Michael Crowder, Oxford, OH (US)

(73) Assignee: MINED XAI LLC, Dayton, OH (US)

(21) Appl. No.: 19/644,848

(22) Filed: Apr. 10, 2026

Related U.S. Application Data
(60) Provisional application No. 63/786,825, filed on Apr. 10, 2025.

(51) Int. Cl. G16C 20/80; G16C 20/70

(52) U.S. Cl. CPC G16C 20/80; G16C 20/70

(57) ABSTRACT

An example method comprises receiving multimodal analysis data including chemical data and sensory data, generating an n-dimensional feature space representation of the multimodal analysis data, projecting the feature space into a manifold embedding using a similarity metric, performing recursive hierarchical decomposition of the manifold embedding using non-overlapping coverings to generate a connected-component network including branch points, performing a local transform operation for leaf nodes of the connected-component network to generate local object embeddings, computing co-enrichment scores between features of different modalities based on co-localization within the local object embeddings, generating a unified multimodal representation based on the co-enrichment scores, and generating a visualization of the unified multimodal representation, the visualization including nodes and relationships representing correlations between the modalities.

### CROSS-REFERENCE TO RELATED APPLICATION

This application claims priority to U.S. Provisional Patent Application No. 63/786,825 filed on Apr. 10, 2025, and entitled “Explainable Machine Learning Systems and Methods for Data Discovery and Insight Generation,” which is incorporated in its entirety herein by reference.

### FIELD OF THE INVENTION(S)

Embodiments of the present invention(s) are generally related to insight discovery using artificial intelligence approaches for report and visualization of insights and in particular, to generating component-connected architectures of underlying data to generate explainable insights.

### BACKGROUND

As the collection and storage of data have increased, there is an increased need to analyze the data for explainable insights. Examples of large datasets may be found in financial services companies, flavor analysis, biotech, and academia. Unfortunately, previous methods of analysis of large multidimensional datasets tend to be insufficient (if possible at all) to identify important relationships.

Previous methods of analysis often use clustering. Clustering is generally too blunt an instrument to identify important relationships in the data (i.e., inherent relationships in the data may be lost within the analysis or noise created by the approach). Similarly, linear regression, projection pursuit, principal component analysis, and multidimensional scaling often do not reveal important relationships. Existing linear algebraic and analytic methods are too sensitive to large-scale distances and, as a result, lose detail.

### SUMMARY

An example non-transitory computer-readable medium comprises executable instructions. The executable instructions may be executable by one or more processors to perform a method. An example method may comprise receiving multimodal analysis data including chemical data and sensory data, generating an n-dimensional feature space representation of the multimodal analysis data, projecting the feature space into a manifold embedding using a similarity metric, performing recursive hierarchical decomposition of the manifold embedding using non-overlapping coverings to generate a connected-component network including branch points, performing a local transform operation for leaf nodes of the connected-component network to generate local object embeddings, computing co-enrichment scores between features of different modalities based on co-localization within the local object embeddings, generating a unified multimodal representation based on the co-enrichment scores, and generating a visualization of the unified multimodal representation, the visualization including nodes and relationships representing correlations between the modalities.

In some embodiments, the chemical data comprises gas chromatography data. The preprocessing may include baseline correction and peak alignment. The embedding may use a nonlinear dimensionality reduction technique.

In various embodiments, recursive hierarchical decomposition may include generating coverings at multiple resolutions. The co-enrichment scores may quantify co-localization of features. The multimodal fusion may include merging connected-component networks.

In some embodiments, the peak alignment includes time-warping. The co-enrichment may be computed using similarity distributions.

In some embodiments, embeddings preserve similarity relationships.

An example system may comprise at least one processor and memory containing instructions. The instructions may be executable by the at least one processor to receive multimodal analysis data including chemical data and sensory data, generate an n-dimensional feature space representation of the multimodal analysis data, project the feature space into a manifold embedding using a similarity metric, perform recursive hierarchical decomposition of the manifold embedding using non-overlapping coverings to generate a connected-component network including branch points, perform a local transform operation for leaf nodes of the connected-component network to generate local object embeddings, compute co-enrichment scores between features of different modalities based on co-localization within the local object embeddings, generate a unified multimodal representation based on the co-enrichment scores, and generate a visualization of the unified multimodal representation, the visualization including nodes and relationships representing correlations between the modalities.

An example method comprises receiving multimodal analysis data including chemical data and sensory data, generating an n-dimensional feature space representation of the multimodal analysis data, projecting the feature space into a manifold embedding using a similarity metric, performing recursive hierarchical decomposition of the manifold embedding using non-overlapping coverings to generate a connected-component network including branch points, performing a local transform operation for leaf nodes of the connected-component network to generate local object embeddings, computing co-enrichment scores between features of different modalities based on co-localization within the local object embeddings, generating a unified multimodal representation based on the co-enrichment scores, and generating a visualization of the unified multimodal representation, the visualization including nodes and relationships representing correlations between the modalities.

An example non-transitory computer-readable medium comprises executable instructions. The executable instructions may be executable by one or more processors to perform a method. An example method may comprise receiving analysis data from at least one data source, projecting the analysis data to a first embedding based on at least one metric, determining a first lowest cover resolution of the first embedding that identifies non-overlapping secondary coverings based on sets within one of the covers of the first embedding, identifying a branch point of a first connected-component network based on the non-overlapping secondary coverings, generating subsets from the branch point based on the non-overlapping secondary coverings, if a network generation threshold has not been met, then for each subset from the branch point, determining a second lowest cover resolution that identifies non-overlapping secondary coverings based on the sets within one of the covers of a particular subset to identify a new branch point and new subsets from that branch point of the first connected-component network, for each leaf of the connected-component network, identify embeddings of a feature space and generate a local object embedding space using the transposition of segmented features with related objects, adding coordinates of objects within each leaf of the local object embedding to a data array, projecting array data from the data array to a second embedding, determining a third lowest cover resolution of the second embedding that identifies non-overlapping secondary coverings based on sets within one of the covers of the second embedding, identifying a branch point of a second connected-component network based on the non-overlapping secondary coverings, generating subsets from the branch point based on the non-overlapping secondary coverings,

if a network generation threshold has not been met, then for each subset from the branch point, determining a second lowest cover resolution that identifies non-overlapping secondary coverings based on the sets within one of the covers of a particular subset to identify a new branch point and new subsets from that branch point of the second connected-component network, and generating a visualization depicted centroids of leaves and branches within the second connected-component network.

The method may further comprise generating the secondary coverings by determining, for each set that has data within the cover, a centroid and determining a radius based on the centroid that covers at least that particular set. The centroid for a particular set may be determined based on the data within that particular set.

The first embedding may comprise a metric space containing projected data, the projected data being one-to-one in the first embedding. The new branch points and new segments may be determined based on new non-overlapping secondary coverings until the network generation threshold is met. In some embodiments, projecting the array data from the data array to the second embedding uses at least the same metric as projecting the received data to the first embedding.

In some embodiments, for each leaf of the first connected-component network, the method comprises projecting the leaf data of that leaf into a separate embedding and determining non-overlapping secondary coverings at the lowest resolution covering of that particular separate embedding to identify metafeature groups. The object membership of each metafeature group of each leaf may be added to the data array. The object membership of each metafeature group of each leaf may be added to the data array before projecting the array data from the data array to the second embedding.

An example system may comprise at least one processor and memory containing instructions. The instructions may be executable by the at least one processor to receive analysis data from at least one data source, project the analysis data to a first embedding based on at least one metric, determine a first lowest cover resolution of the first embedding that identifies non-overlapping secondary coverings based on sets within one of the covers of the first embedding, identify a branch point of a first connected-component network based on the non-overlapping secondary coverings, generate subsets from the branch point based on the non-overlapping secondary coverings, if a network generation threshold has not been met, then for each subset from the branch point, determine a second lowest cover resolution that identifies non-overlapping secondary coverings based on the sets within one of the covers of a particular subset to identify a new branch point and new subsets from that branch point of the first connected-component network, for each leaf of the connected-component network, identify embeddings of a feature space and generate a local object embedding space using the transposition of segmented features with related objects, add coordinates of objects within each leaf of the local object embedding to a data array, project array data from the data array to a second embedding, determine a third lowest cover resolution of the second embedding that identifies non-overlapping secondary coverings based on sets within one of the covers of the second embedding, identify a branch point of a second connected-component network based on the non-overlapping secondary coverings, generate subsets from the branch point based on the non-overlapping secondary coverings, if a network generation threshold has not been met, then for each subset from the branch point, determine a second lowest cover resolution that identifies non-overlapping secondary coverings based on the sets within one of the covers of a particular subset to identify a new branch point and new subsets from that branch point of the second connected-component network, and generate a visualization depicted centroids of leaves and branches within the second connected-component network.

An example method may comprise receiving analysis data from at least one data source, projecting the analysis data to a first embedding based on at least one metric, determining a first lowest cover resolution of the first embedding that identifies non-overlapping secondary coverings based on sets within one of the covers of the first embedding, identifying a branch point of a first connected-component network based on the non-overlapping secondary coverings, generating subsets from the branch point based on the non-overlapping secondary coverings, if a network generation threshold has not been met, then for each subset from the branch point, determining a second lowest cover resolution that identifies non-overlapping secondary coverings based on the sets within one of the covers of a particular subset to identify a new branch point and new subsets from that branch point of the first connected-component network, for each leaf of the connected-component network, identify embeddings of a feature space and generate a local object embedding space using the transposition of segmented features with related objects, adding coordinates of objects within each leaf of the local object embedding to a data array, projecting array data from the data array to a second embedding, determining a third lowest cover resolution of the second embedding that identifies non-overlapping secondary coverings based on sets within one of the covers of the second embedding, identifying a branch point of a second connected-component network based on the non-overlapping secondary coverings, generating subsets from the branch point based on the non-overlapping secondary coverings, if a network generation threshold has not been met, then for each subset from the branch point, determining a second lowest cover resolution that identifies non-overlapping secondary coverings based on the sets within one of the covers of a particular subset to identify a new branch point and new subsets from that branch point of the second connected-component network, and generating a visualization depicted centroids of leaves and branches within the second connected-component network.

### BRIEF DESCRIPTION OF THE DRAWINGS

FIG. 1 depicts an overview of construction of a network for explanation generation in some embodiments.

FIG. 2 depicts an example environment for an explainable machine learning system in some embodiments.

FIG. 3 depicts a block diagram of an explainable machine learning system in some embodiments.

FIG. 4A depicts a method for generating explainable insights using component-connected architecture(s) in some embodiments.

FIG. 4B depicts a method for generating component-connected architectures in some embodiments.

FIG. 5 depicts an initial approximation of the recursive hierarchical decomposition (RHD) of the dataset in some embodiments.

FIG. 6 depicts a tower of covers utilizing stepwise resolution increases in some embodiments.

FIG. 7 depicts subspace clustering of feature space in some embodiments.

FIG. 8 depicts a recursive hierarchical decomposition (e.g., a first connected-component network) of the feature space in some embodiments.

FIG. 9 depicts a reference frame context in some embodiments.

FIG. 10 depicts a second layer of the network (e.g., DTNN) in some embodiments.

FIG. 11 depicts the optional creation of metafeatures to explain segmentation in some embodiments.

FIG. 12 depicts the explanation element showing group membership for a local feature space in some embodiments.

FIG. 13 depicts the utilization of local embedding coordinates as novel features across the local transposed elements in some embodiments.

FIG. 14 depicts local clustering group information that is encoded as membership to hierarchically defined groups in some embodiments.

FIG. 15 depicts creation of an RHD summary in some embodiments.

FIG. 16 depicts leaf node centroids of global object space RHD in some embodiments.

FIG. 17 depicts subset node centroids of global object space RHD 1704 in some embodiments.

FIG. 18 depicts leaf and subset centroid placements within the embedding for each layer of the RHD in some embodiments.

FIG. 19 depicts a topological summary of global object space RHD 1902 in some embodiments.

FIG. 20 depicts an interactive visualization of the topological summary in some embodiments.

FIG. 21 depicts a statistical feature and metafeature summary of the RHD leaf node in some embodiments.

FIG. 22 depicts the hybrid EET1/EET2 showing group membership for selected local object space feature(s) in some embodiments.

FIG. 23 depicts an interactive visualization in some embodiments.

FIG. 24 is an example of a feature description for bourbons that is derived from the feature space RHD.

FIG. 25 depicts an example UMAP pre-embedding (HDBSCAN clusters).

FIG. 26 depicts an example UMAP pre-embedding (run order).

FIG. 27 depicts an example UMAP post-embedding (HDBSCAN clusters).

FIG. 28 depicts an example UMAP post-embedding (run order).

FIG. 29 depicts an example original spectra (control sequence). In this example, intensity is along the y-axis and retention time normalized spectra is along the x-axis.

FIG. 30 depicts an example retention time normalized spectra (run order) along the x-axis and intensity along the y-axis.

FIG. 31 depicts an example retention time normalized spectra (run order) along the x-axis and intensity along the y-axis.

FIG. 32 depicts an example retention time along the x-axis and intensity along the y-axis.

FIG. 33 depicts example spectra quotients (e.g., from the gas chromatograph). Median quotients are along the y-axis and the retention time (median quotient regression) is along the x-axis.

FIG. 34 depicts an example graph of SOP and control with the median quotient along the y-axis and the barrel number (e.g., for the specific barrel of bourbon) along the x-axis.

FIG. 35 depicts an example wheat-to-rye graph.

FIG. 36 depicts a proof 105 to proof 125 graph.

FIG. 37 depicts an example graph for seasoning of staves (e.g., comparison of 6 to 12 months).

FIG. 38 depicts an example graph of grain comparison (e.g., tight, average, coarse, and control).

FIG. 39 depicts an example storage graph for comparison of wooden to concrete).

FIG. 40 depicts an example char graph for comparing type #3 char to type #4 char.

FIG. 41 depicts an example tree graph for comparison of top of tree to bottom of tree.

FIG. 42 depicts an example ring graph for comparison of the number of rings.

FIG. 43 depicts an example distill graph for comparison of different distillation dates.

FIG. 44 depicts a run order graph for a comparison of different run orders.

FIG. 45 depicts a block diagram of an example digital device 4500 according to some embodiments.

FIG. 46 depicts an explainable machine learning system in some embodiments.

FIG. 47 depicts workflow overviews used in the analysis of the Single Oak Project in some embodiments.

FIG. 48 depicts feature clusters in some embodiments.

FIG. 49 depicts an object space topological hierarchical decomposition in some embodiments.

FIG. 50 depicts examples of enrichment scores measured in a local transform embedding in some embodiments.

FIG. 51A depicts an example of query scores overlaid on a metadata feature summary embedding showing which neighborhoods of metadata features pertain most directly to a given metadata feature (left).

FIG. 51B depicts an example co-activation embedding of chemical and production object space THD clusterings (top left), colored by depth in an “activation path” from the root node to an example leaf node of the merge THD.

FIGS. 52A and B show some of the results obtained from the example calculation of query matrices for production and sensory metadata as well as production and consumer preference metadata.

Throughout the drawings, like reference numerals will be understood to refer to like parts, components, and structures.

### DETAILED DESCRIPTION

As discussed herein, various embodiments of systems and methods include generation of a component-connected architecture. Components of the component-connected architecture may define features, feature/object metadata, and/or object relationships. The component-connected architecture may enable the discovery of relationships of features within high-dimensional spaces.

In one example of the component-connected architecture, dimensionality-reduced feature sets are used to create a local transpose of the isolated features to derive local relationships of the objects within the feature space. A hierarchical representation of the objects may be generated using the local transpose embedding coordinates that feed into the object space hierarchical understanding to create topological summaries of hierarchical information. The topological summaries of hierarchical information may provide explanation information (e.g., through generation of new component-connected architectures across subsets of the previous component-connected architecture). The explanation information suggests or explains relationships within the underlying data.

An interactive visualization may be optionally generated to enable selection of data within the topological summaries of hierarchical information and/or statistical interrogation to display explainable information of complex relationships at a simplified lower Dimensional representation. The interactive visualization may, in some embodiments, enable annotation.

Alternatively for additionally, reports may be generated that includes topological summaries of hierarchical information and/or statistical data explaining complex relationships at a simplified lower dimensional representation.

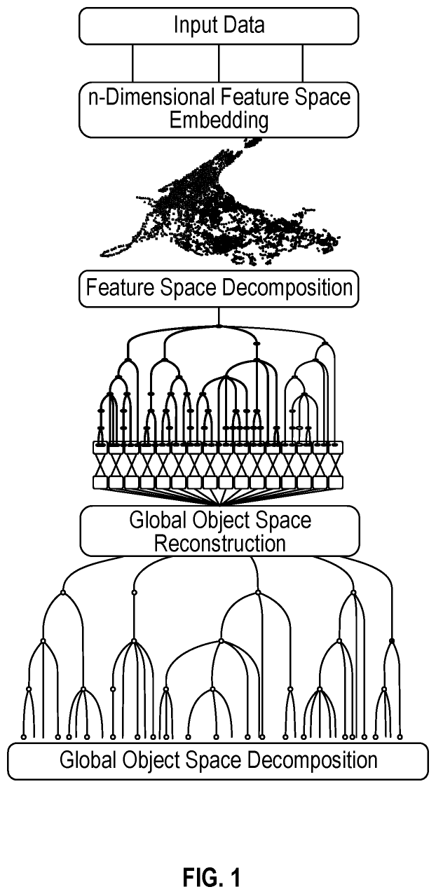

FIG. 1 depicts an overview of construction of a network for explanation generation in some embodiments. In various embodiments, an explainable machine learning system constructs a network (e.g., a deep topological neural network (DTNN)) for automating explainable machine learning methods for data discovery and insight generation. Utilizing topological data analysis and hierarchical processing methods, the explainable machine learning system constructs a component-connected architecture that:
(i) discovers relationships of features in high-dimensional spaces,
(ii) utilizes dimensionality-reduced feature sets to create a local transpose of the isolated features to derive local relationships of the objects within the feature space, and
(iii) formulates a hierarchical representation of the objects using the local transpose embedding coordinates that feed into a complete object space hierarchical understanding.

The explainable machine learning system may create methods for hierarchically structuring information and creating topological summaries of hierarchical information for explanation generation. As discussed herein, the overall process may create components for defining features, feature/object metadata, and object relationships that enable automated processing, statistical interrogation, and/or explainable demonstration of complex relationships at a simplified lower dimensional representation for human evaluation and annotation. In some embodiments, as opposed to competing methods, the explainable machine learning system may establish embedded metafeatures created within the layers of the neural network to contribute to machine learning explainability.

It will be appreciated that the representation may or may not be visualized.

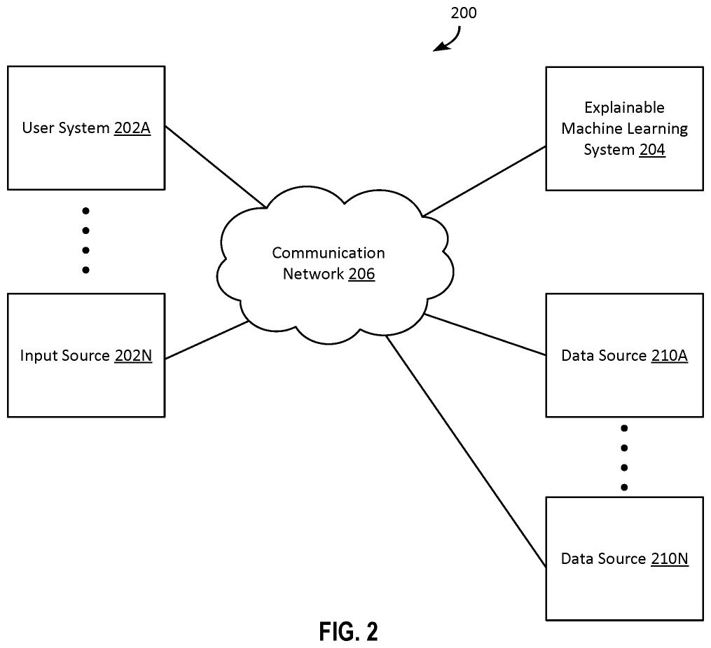

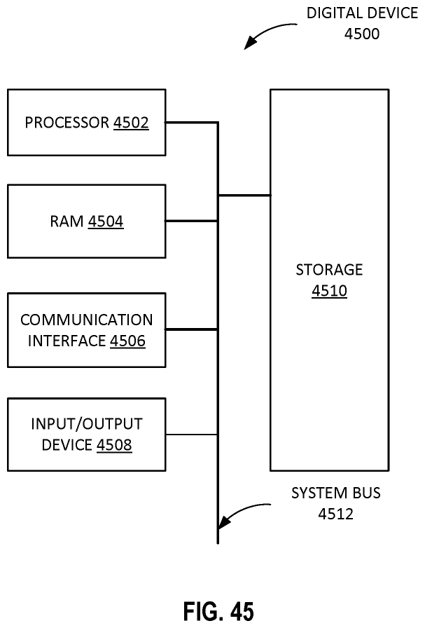

FIG. 2 depicts an example environment 200 for an explainable machine learning system in some embodiments. The example environment 200 includes an explainable machine learning system 204, user systems 202A-N, data sources 210A-N, and a communication network 206. Each of the explainable machine learning system 204, user systems 202A-N, and data sources 210A-N may be or include any number of digital devices. A digital device is any device with at least one processor and memory. Digital devices are further discussed herein, for example, with reference to FIG. 45.

The explainable machine learning system 204 may receive data from any number of data sources for analysis as generally discussed with reference to FIG. 1. The explainable machine learning system 204 may retrieve information, prepare the information for analysis, identify segments of data that preserve and/or highlight significant features, determine features/meta-features for embedding, and identify explainable elements. Explainable machine learning system 204 may further generate a visualization or generate a report to display information and insights. In some embodiments, the visualization may be interactive thereby allowing users to make selections of nodes (centroids) of the generated networks. The interactive visualization is further discussed herein.

One or more of the user systems 202A-N may display interfaces to a user that the user may utilize to control the explainable machine learning system 204. For example, a user of the user system 202A may provide instructions to identify data retained by data sources 210A-N for retrieval, provide metrics/filters, and inspect insights and visualizations from the explainable machine learning system 204.

One or more of the data sources 210A-N may retain information for analysis by the explainable machine learning system 204. In some embodiments, the explainable machine learning system 204 may provide transformed databases, tables, analysis, reports, and/or the like to any number of the data sources 210A-N. In some examples, the data sources 210A-N may include data warehouses, data links, cloud storage, local storage, or any combination thereof.

In some embodiments, the communication network 206 may represent one or more computer networks (for example, LAN, WAN, and/or the like). The communication network 206 may provide communication between any of the explainable machine learning system 204, user systems 202A-N, and/or data sources 210A-N. In some implementations, the communication network 206 comprises computer devices, routers, cables, uses, and/or other network topologies. In some embodiments, the communication network 206 may be wired and/or wireless. In various embodiments, the communication network 206 may comprise the Internet, one or more networks that may be public, private, IP-based, non-IP based, and so forth.

It will be appreciated that any number of unrelated users (e.g., users from different and unrelated enterprises, commercial entities, research institutions, governments, and/or the like) perform analysis on unrelated data sets from any number of data sources by the same explainable machine learning system 204. In some embodiments, explainable machine learning system 204 may provide insights and analysis on a variety of different data sets on behalf of any number of different users.

In various environments, a particular user with privileged data rights to confidential information may provide the information (e.g., encrypted, protected, unprotected, and/or the like) for analysis by the explainable machine learning system 204. The explainable machine learning system 204 may maintain a record of all actions performed on the database, stored any information related to the analysis of the original data within required unprotected data storage, and or authenticate users or devices as required.

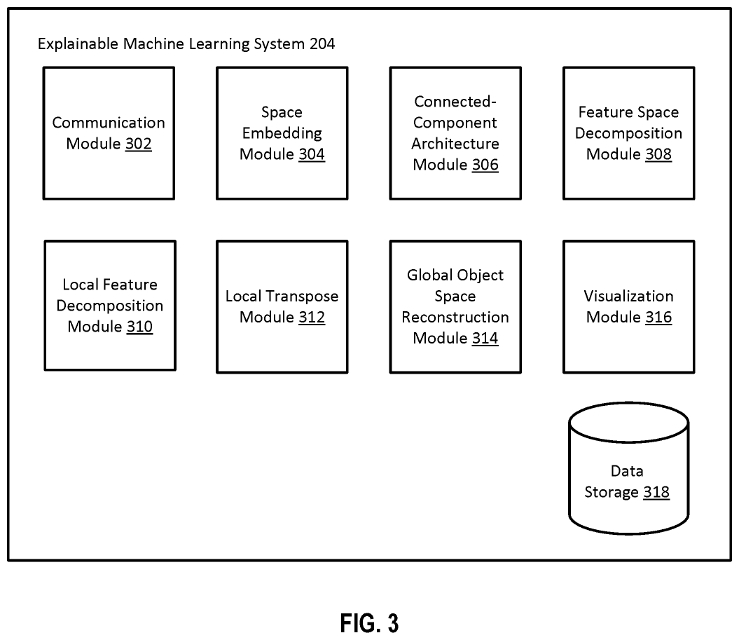

FIG. 3 depicts a block diagram of an explainable machine learning system 204 in some embodiments. The explainable machine learning system 204 comprises a communication module 302, a space embedding module 304, a connected-component network module 306, a feature space decomposition module 308, a local feature decomposition module 310, a local transpose module 312, a global object space reconstruction module 314, a visualization module 316, and a data storage 318.

The communication module 302 may send and/or receive requests and/or data from the data source(s) 110A-110N and/or user devices 102A-N. In one example, the communication module 302 receives data to be analyzed from data source 110A.

The communication module 302 may receive requests and/or data from the user system 106, the input source system 108, and the output destination system 110. The communication module 302 may also send requests and/or data to the user system 106, the input source system 108, and the output destination system 110.

The communication module 302 may receive or provide data or requests to any of the modules of the explainable machine learning system 204. In some buttons, the communication module 302 may receive or provide data to the user devices 102A-N and/or data sources 110A-N.

In various embodiments, the communication module 302 receives or retrieves n-dimensional matrix. The n-dimensional matrix may be any data from any number of data sources. In various embodiments, the communication module 302 retrieves data from two or more different data sources 110A-N. The communication module 302 may combine the data from the different data sources to generate the n-dimensional matrix.

The feature space embedding module 304 may generate a lower dimensional embedding feature space by projecting the data based on metrics and/or filters discussed herein.

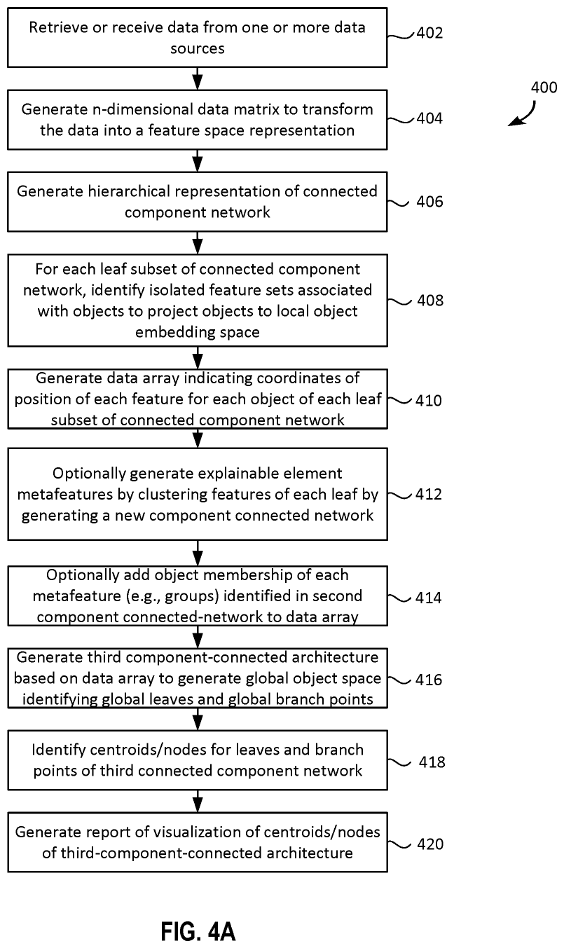

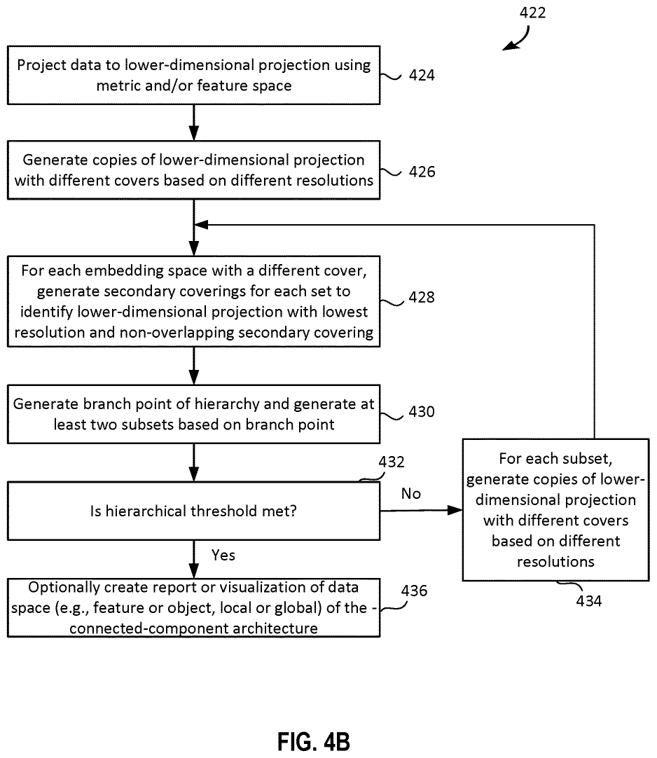

The connected-component network module 306 may generate connected-component networks (e.g., using the “tower of covers” approach discussed herein). The process is discussed with regard to FIG. 4B.

The feature space decomposition module 308 may generate a lower dimensional embedding of the feature space as described herein for each leaf of the first connected-component network as described herein.

The connected-component network module 306 may identify segment (branch) points of the embedded space at different thresholds. The subset of connected components (e.g., derived from the tower covers) may create data subsets for repeating (e.g., nested) above method to produce a hierarchy of local feature sets of common similarity measures. As a result, a recursive hierarchical decomposition (RHD) of the feature space is generated.

In some embodiments, the local features of the RHD group subsets can be visualized back within their reference frame, establishing an explanatory element.

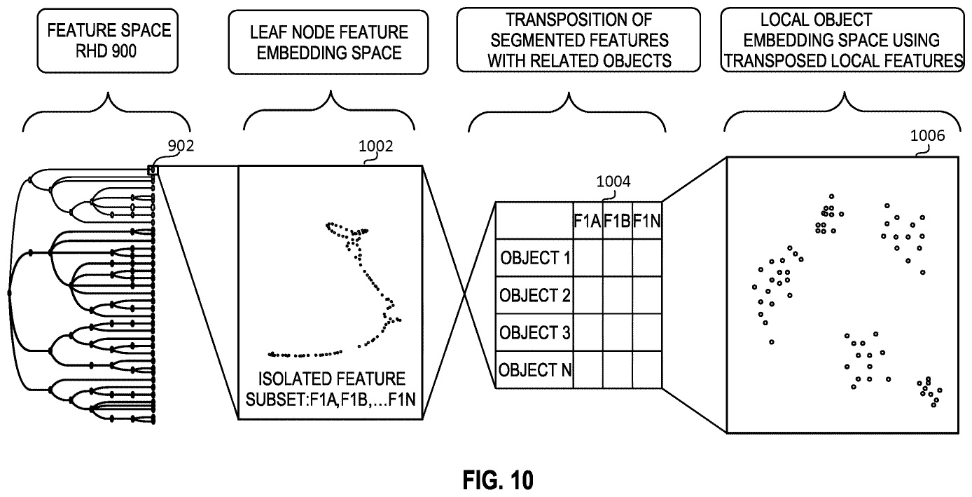

The local feature decomposition module 310 may assist in identifying features in individual leaves of the feature space for embedding in the leaf node feature embedding space or generating the local object embedding space used to transpose local features as discussed with regard to FIG. 10.

The local transpose module 312 is configured to locally transpose the RHD isolated feature sets (e.g., objects as rows and RHD isolated features as columns) as discussed herein.

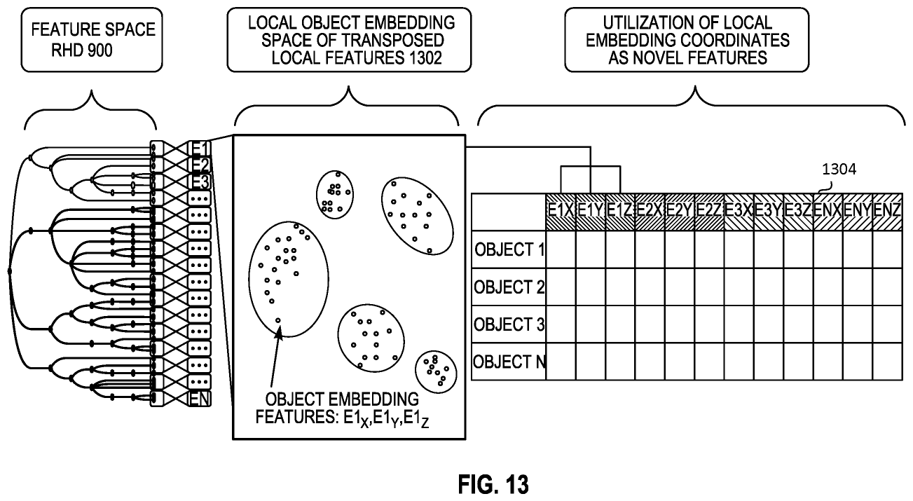

The global object space reconstruction module 314 may generate the global object space, the top node embedding of the global object space RHD 1504, and/or the topological summary of global object space RHD 1902 as described with regard to FIG. 13-19.

FIG. 3 depicts an example method for explainable analysis in some embodiments. The steps of the method depicted in FIG. 3 will be further described in FIGS. 4-23.

As discussed herein, various embodiments of systems and methods include generation of a component-connected architecture. The component-connected architecture may enable the discovery of relationships of features within high-dimensional spaces.

FIG. 4A depicts a method for generating explainable insights using component-connected architecture(s) in some embodiments.

In step 402, the communication module 302 retrieves or receives data from one or more data sources (e.g., data sources 110A-N). The data may be in any form or organization.

In step 404, the communication module 302 and/or the feature space embedding module 304 may generate an n-dimensional data matrix to transform the data into a feature space representation.

The feature space representation may include features as rows and objects as columns. In various embodiments, the communication module 302 may perform processing on any of the data received from the data sources. For example, the communication module 302 may normalize data, create new features, perform calculations to generate new features, and/or the like. In another example, the communication module 302 may convert data received from one or more data sources into the feature space representation (e.g., features as rows and objects as columns). In some embodiments, the communication module 302 may combine data sets from any number of data sources once each of the data sets are in the feature space representation.

In step 406, the explainable machine learning system tool for may generate a connected-component architecture and a hierarchical representation of the first component-connected architecture based on the feature space representation of the data received from the data sources or user devices. FIG. 4B depicts a method for generating a connected architecture.

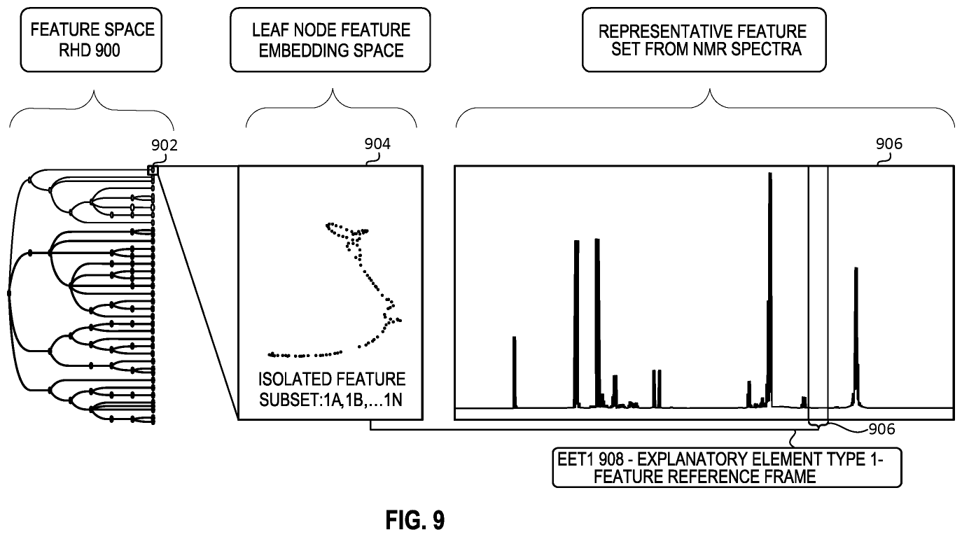

After the first connected-component network is generated based on the feature space representation, in step 408, for each leaf subset of the connected component network, the feature space decomposition module 308 may identify isolated feature sets the social of objects and/or project those objects to a local object embedding space. This process is discussed with regard to FIGS. 9 and 10.

Each leaf (e.g., leaf node) identifies an embedding of the feature space. For example, a leaf node may include an isolated featured subset. The isolated featured subset may be used to generate a transposition of segmented features with related objects. In this example, each row includes the original objects and columns are for each feature of the isolated featured subset for that leaf.

In step 410, the feature space decomposition module 308, the local feature decomposition module 310, or the local transpose module 312 may generate a data array indicating coordinates of a position of each feature for each object of each leaf subset of the connected component network. This process is further discussed with regard to FIGS. 10 and 13.

In step 412, the local transpose module 312 may optionally generate explainable element meta-features by clustering features of each leaf. In one example, a local object embedding space may be generated using the transposition of segmented features with related objects. In one example, metrics and/or filters (e.g., the same metrics and/or filters used to generate one or more other projections) may be used to project the objects into the local object embedding space.

For each leaf node, a coordinate position of an object in its related local object embedding space is identified and included in the data array. The data array includes rows of objects as well as columns identifying coordinates of that object in each local object embedding space of one or more (e.g., all) leaf nodes.

For optional step 412, another component connected architecture using the methodologies described herein may be created for each local object embedding space to identify clusters or groups within the local object embedding space. For example, different coverings can be applied to one or more embedding spaces to identify nonoverlapping secondary coverings (e.g., using the methods described herein). The nonoverlapping secondary coverings identify subset branch points and two or more subsets within the embedding space may be similarly assessed (e.g., for each subset from the branch point, different covers can be applied to identify nonoverlapping secondary coverings to further identify branch points for further analysis) until a threshold is reached. The threshold may be any limiting determination of function including, for example, a number of subsets found, a statistical measure based on the original data set, a number of groups based on the data within the local object embedding space, and/or the like.

In this optional example, an object may be a member of a group which may be termed as a meta-feature.

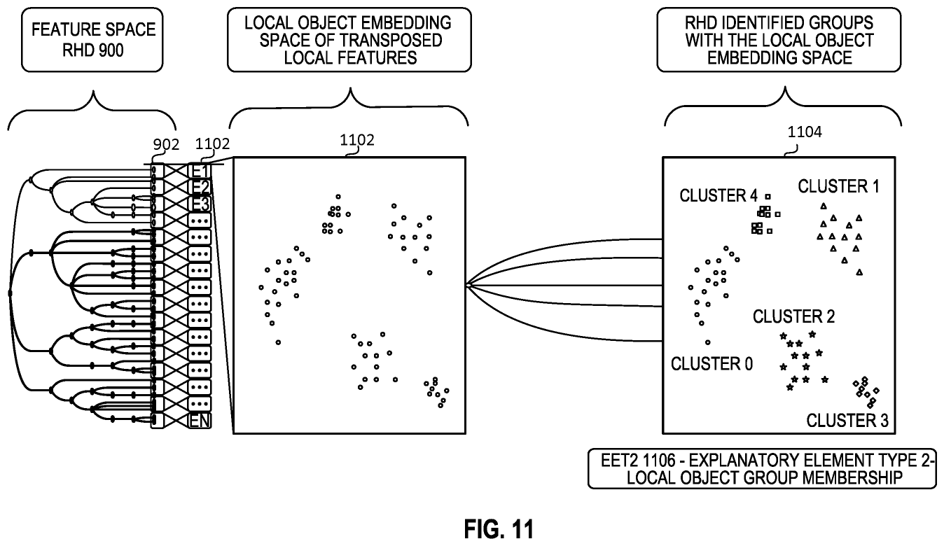

In step 414, each meta-feature may be uniquely identified (e.g., MF1-N) for each local space and membership of that meta-feature group for each object across all local embedding spaces may be added to the data array (e.g., the same data array that contains object coordinates across the leaves of the first connected-component network). This process is further described with reference to FIGS. 11-14.

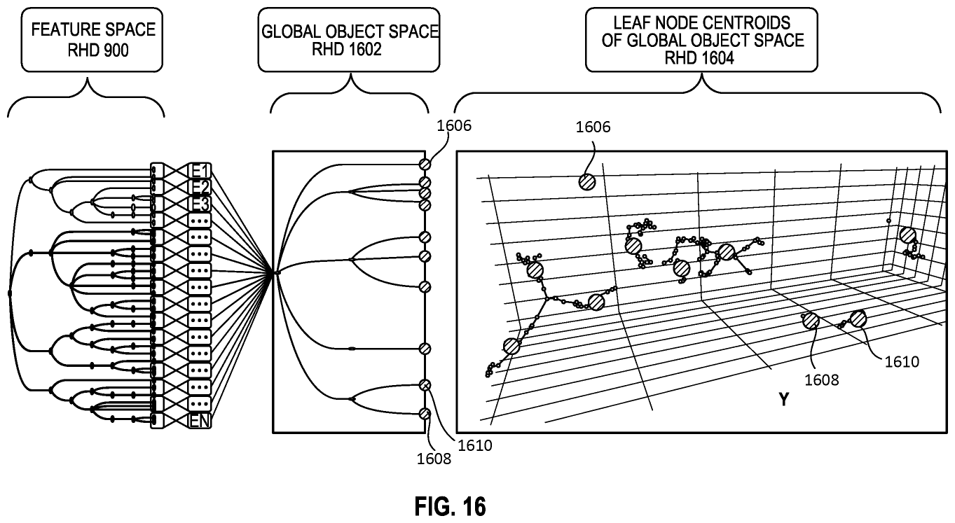

In step 416, the connected-component network module 306 may generate a third connected-component network based on the data array from step 410 or steps 410-414 (e.g., including or not including the metafeatures described herein) to generate a global object space that includes global leaves and global branch points. This process is similar to that described with regard to FIG. 4B but utilizes the data array. This process is further described with reference to FIG. 16.

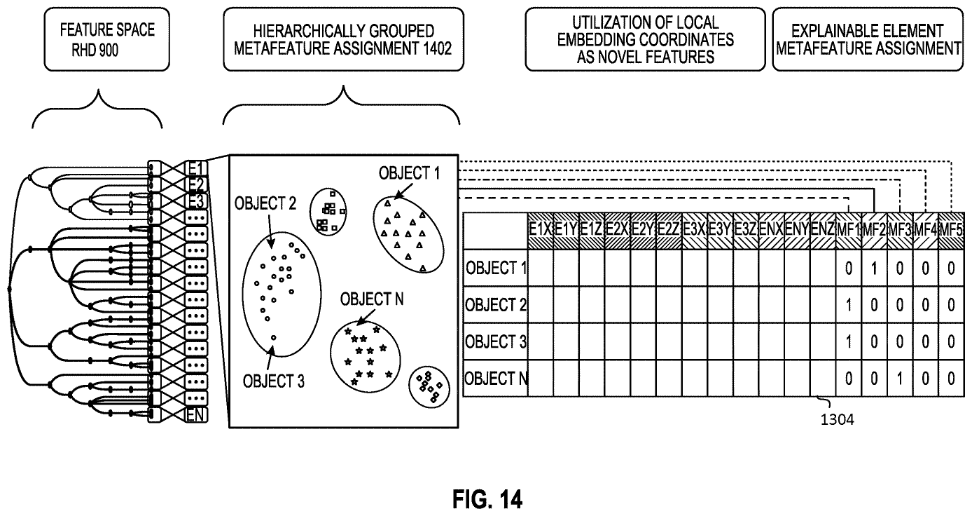

In step 418, the global object space reconstruction module 314 identifies centroids (i.e., nodes) for leaves and branch points of the third connected-component network. This process is further described with regard to FIGS. 14-20.

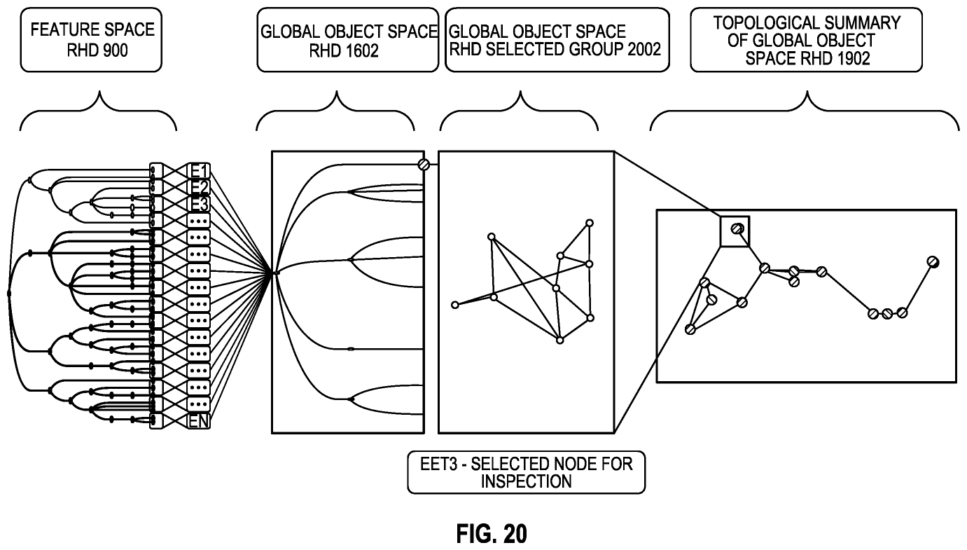

In step 420, the visualization module 316 may generate a report or visualization of the centroids (e.g., nodes) of the third connected-component network (e.g., as depicted in FIG. 20). In some embodiments, the visualization module 316 may generate an interactive visualization interactive visualization to enable selection of data within the topological summaries of hierarchical information and/or statistical interrogation to display explainable information of complex relationships at a simplified lower Dimensional representation. The interactive visualization may, in some embodiments, enable annotation.

Alternatively, for additionally, reports may be generated that includes topological summaries of hierarchical information and/or statistical data explaining complex relationships at a simplified lower dimensional representation.

FIG. 4B depicts a method for generating component-connected networks in some embodiments. It will be appreciated that this process may be titled a “tower of covers” approach to network generation.

In step 424, the space embedding module 304 may project data from the received data (e.g., from the feature space representation or data array discussed herein) into an embedding space. The space embedding module 304 may project the data using any number of ways. For example, the space embedding module 304 may utilize one or more metrics and/or filters (e.g., receipt from the user device) to make the projection.

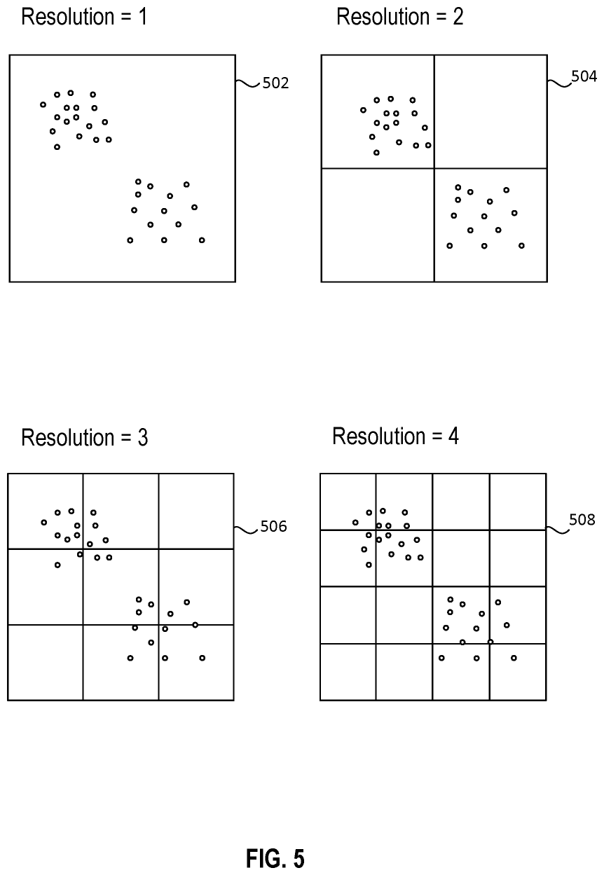

The connected-component network module 306 may perform steps 426 through 444 to generate the connected-component network. In step 426, the connected-component network module 306 may apply different covers of the embedding space to identify nonoverlapping secondary coverings for branch identification. The connected-component network module 306 may generate sequentially apply each different covering to the embedding space and/or generate copies of the embedding space and apply a different covering to each of the embedding spaces. FIG. 5 includes an example of the different coverings applied to the same embedding space (e.g., the projection of the data generated in step 424).

It will be appreciated that each cover may create one or more sets (e.g., individual squares covering the embedding space as depicted in FIG. 5).

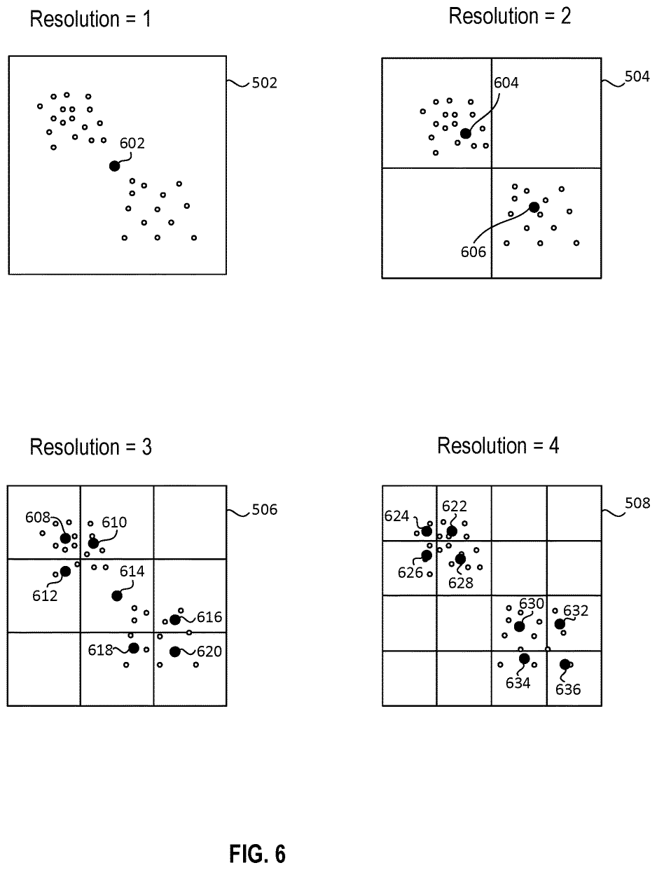

In step 428, for each embedding space with a different cover, the connected-component network module 306 generates secondary coverings for each set to identify the lower dimensional projection with the lowest resolution and nonoverlapping secondary coverings. In one example, a centroid is determined for each set within the covering. The centroid is determined based on the data within that set as discussed herein. This process is discussed with regard to FIG. 6.

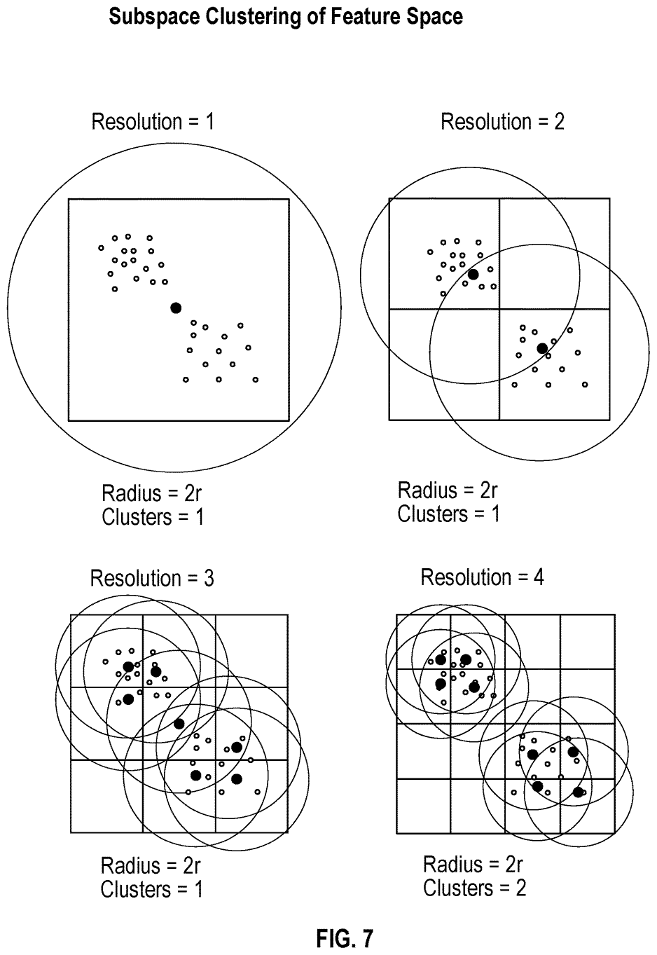

Brief centroid secondary coverings generated using the centroid at the center of the secondary covering. The secondary covering covers the particular set of data points. The connected-component network module 306 determines if there is overlap between the two secondary coverings (e.g., if there are separate clusters). A branch point is identified based on the embedding space with the lowest resolution that has at least two data sets with nonoverlapping secondary covers. This process is further discussed with regard to FIG. 7.

In some embodiments, to generate the first component-connected architecture, dimensionality-reduced feature sets are used to create a local transpose of the isolated features to derive local relationships of the objects within the feature space. A hierarchical representation of the objects may be generated using a local transpose embedding coordinates that feed into the object space hierarchical understanding to create topological summaries of hierarchical information. The topological summaries of hierarchical information may provide explanation information. The explanation information suggests or explains relationships within the underlying data.

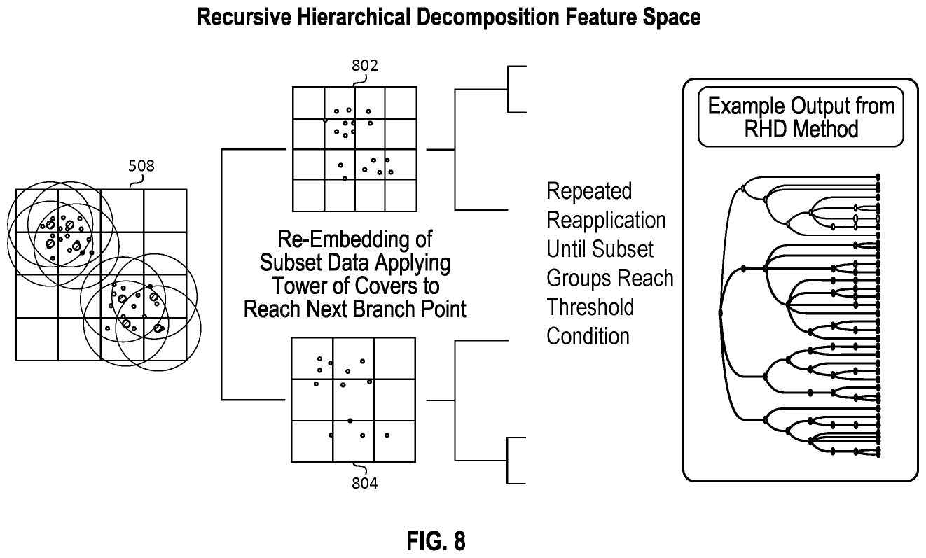

In step 430, the connected-component network module 306 generates a branch point of the hierarchy based on the projection with the lowest resolution and nonoverlapping secondary covering. The connected-component network module 306 generates at least two subsets based on the branch point. This process is further discussed with regard to FIG. 8.

In step 432, the connected-component network module 306 determines if a hierarchical threshold is met to terminate the network generation process. It will be appreciated that there may be any number of thresholds to generate the network generation process as discussed herein. The network will continue to be generated with additional branch points and subsets until the hierarchical threshold is met.

If the hierarchical threshold is not met, the method continues to step 434. In step 434, in a manner similar to that of step 426, for each subset of the branch, the connected-component network module 306 applies different covers to each subset to identify the lowest resolution with nonoverlapping secondary coverings. The method continues to step 428 as applied to each subset from the branch point.

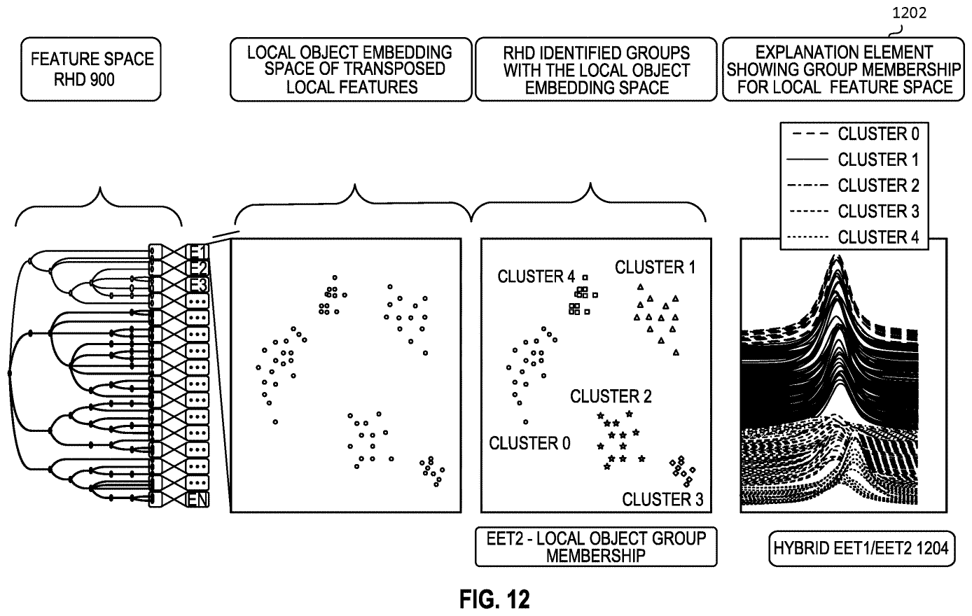

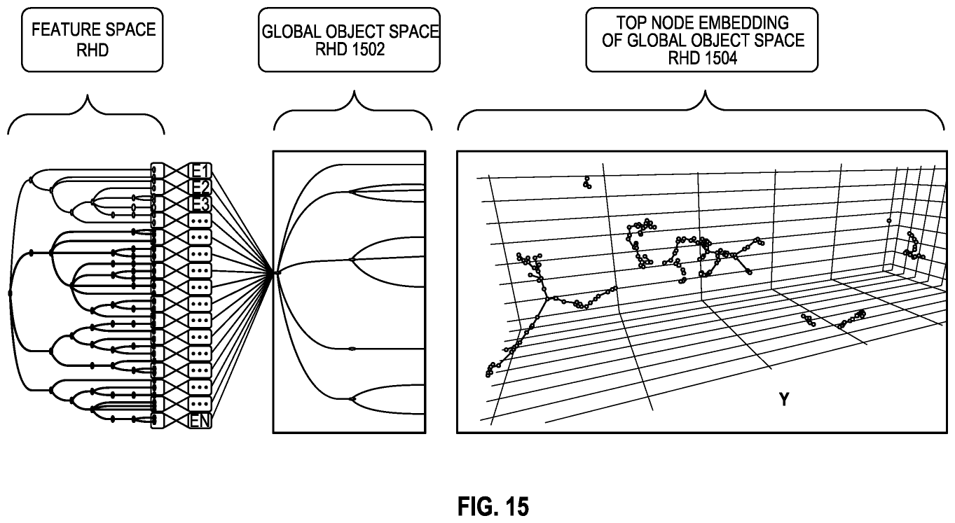

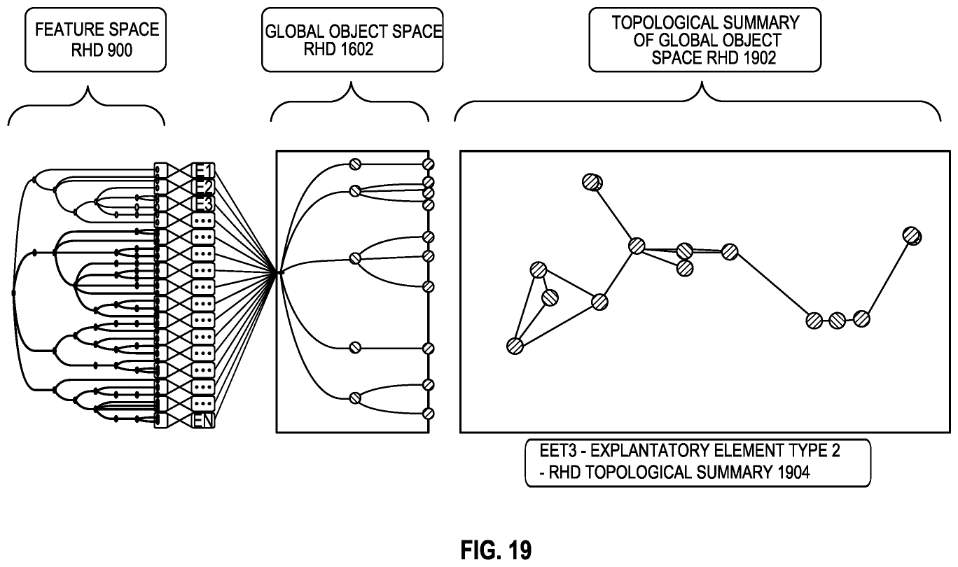

If the hierarchical threshold is met, then the method continues to step 436. In step 436, the connected-component network module 306 and/or the visualization module 316 may optionally generate a report visualization of the resulting data space (e.g., feature or object, local or global) of a connected-component architecture (e.g., the feature space RHD 900 of FIG. 9, the leaf node feature embedding space 1002, explanation element showing group membership from local feature space 1202 in FIG. 12, the top note embedding global object space RHD 1504 in FIG. 15, the global object space RHD 1502 in FIG. 15, the topological summary of global object space RHD 1902 in FIG. 19, and/or the like).

FIG. 5 depicts an initial approximation of the recursive hierarchical decomposition (RHD) of the dataset in some embodiments. Although the term “recursive” is used with respect to the RHD, it will be appreciated that a series of covers may be applied to the same data projection (e.g., the same embedding space) to identify non-overlapping secondary coverings. The process may not be recursive in other aspects as will be shown herein.

In FIG. 5, the initial approximation of the RHD begins through transformation of a high-dimensional data space into a lower dimension projection using any generic dimensionality reduction method combining metric and filter spaces (e.g., Euclidean, cosine, correlation, t-sne, umap, mds, pca, or the like). In one example, the feature space embedding module 304 utilizes a metric and/or filter functions (e.g., spaces) to transform high dimensional data received from the data source into a lower dimensional projection.

Following embedding, the feature space decomposition module 308 may apply a uniform (or non-uniform) cover to the embedding. FIG. 5 depicts a variety of different uniform covers at different resolutions that cover the same data space. Graph 502 depicts a cover (e.g., a square) with a resolution of one that covers the projected data points. Graph 504 depicts a cover of a resolution of 2 (e.g., 2² or 4 uniform squares or sets) covering the data space. Graph 506 depicts a cover of a resolution of 3 (e.g., 3² or 9 uniform squares or sets) covering the data space. Graph 508 depicts a cover of a resolution of 3 (e.g., 4² or 16 uniform squares or sets) covering the data space.

It will be appreciated that a single data space utilizing covers of a specific resolution can be utilized in conjunction with systems of methods discussed herein. Ultimately, in some embodiments, any number of different resolutions may be utilized. Although FIG. 5 depicts square sets, it will be appreciated that the sets of the coverings may be of any shape or combination of shapes (e.g., different intervals and/or sizes).

For 2- and 3-component embeddings, a uniform embedding can be applied as squares, rectangles, or voxels where resolution is defined by the maximum and minimum components in their respective projection space. It is not necessary to preserve any relationship between individual component resolution values and they can be treated as independent parameters. For ease, FIG. 5 depicts the embedded data covered by a uniform square cover at multiple resolutions, although it will be appreciated that any cover at any resolution may be utilized.

The cover will assist with the clustering of the feature space for recursive hierarchical decomposition. FIG. 6 depicts a tower of covers utilizing stepwise resolution increases in some embodiments. The covering of the data includes a subset or plurality of subsets of the data used to compute a centroid of the component values. The centroid may be calculated in any number of ways. In one example, a centroid is calculated based on an average of the data within the individual set of the cover.

In graph 502 of FIG. 6, the centroid is at the center of the data space. In this example, the centroid is represented as point 602 and is calculated based on the data points mapped to the data space (e.g., the metric space). In graph 504 of FIG. 6, for the cover has a resolution of 2, the centroid is calculated based on the data within the individual set (e.g., the individual portion of the cover). In this example, there are two individual sets that are empty (i.e., devoid of mapped data points), and as a result, do not have a centroid. Centroid 604 and centroid 606 are based on the data points contained in their particular sets, respectively. For example, centroid 604 is based on data points contained within its particular set (i.e., the square) but not the data points in any other set of the same space. Similarly, centroid 606 is based on data points contained within its particular set (i.e., the square) but not the data points in any other set.

In graph 506, the data space is divided into nine sets (e.g., graph 506 has a resolution of three). Two of the nine sets have no data points mapped to those individual spaces and therefore have no centroids. Centroids 608, 610, 612, 614, 616, 618, and 620 are each based on the data points within their respective sets.

In graph 508, the data space is divided into 16 sets (e.g., graph 508 has a resolution of four). Eight of the 16 coverings have no data points mapped to those individual spaces and therefore have no centroids. Centroids 622, 624, 626, 628, 630, 632, 634, and 636 are each based on the data points within the respective sets.

FIG. 7 depicts subspace clustering of feature space in some embodiments. Branch points for the non-overlapping connected neighborhood graphs may be identified based on identification of non-overlapping connected neighborhood graphs at the lowest resolution.

In various embodiments, following centroid determination, a circle with a radius of fixed length is centered on each centroid creating a secondary covering. The radius may, for example, be the distance from the centroid to cover the set (e.g., a corner of that set as depicted). Each circle can be parameterized to include a single radius, or a plurality of radii, of differing lengths that scale proportionally to the resolution size.

In FIG. 7, each data space (e.g., graph 502, 504, 506, and 508) is covered by a secondary covering defined by a radius with the respective centroid as the center.

In other words, FIG. 7 demonstrates covering of the embedded space over 4 resolutions. At resolution=1, 2, and 3, non-empty sets result in a single connected component due to a minimum of one centroid being common to a fully connected intersection. At resolution=4, two non-overlapping connected neighborhood graphs (e.g., there are two non-overlapping secondary coverings) are created resulting in a branch point within the topological hierarchy.

Graph 502 in FIG. 7 depicts a single secondary covering that covers the entire embedded space. The secondary covering is based on a radius from the centroid 602 and extends across the covering (which in this case, there is a resolution of one, includes the entire embedded space). As a result, a single cluster (i.e., a cluster=1) is identified.

Graph 504, which has a resolution of two, includes two secondary coverings based on the two centroids 604 and 606, respectively. Since these secondary coverings overlap, a branch point is not identified. Like graph 502, graph 504 has a single cluster (i.e., a cluster=1).

Graph 506 has a resolution of three. As discussed herein, each centroid (e.g., centroid 608, 610, 612, 614, 616, 618, and 620) is the center of its own respective secondary covering. Since these secondary coverings overlap, a branch point is not identified. Like graphs 502 and 504, graph 506 has a single cluster (i.e., a cluster=1).

Graph 508 has a resolution of four. Each centroid (e.g., centroids 622, 624, 626, 628, 630, 632, 634, and 636) is the center of its own respective secondary covering. Here, there are at least two secondary coverings that do not overlap and a branch point is identified. In this example, there are two clusters (i.e., clusters=2).

FIG. 8 depicts a recursive hierarchical decomposition (e.g., a first connected-component network) of the feature space in some embodiments. At each branch point, two or more distinct subsets of the embedded data are created. Each subset is individually re-embedded utilizing a common metric/filter combination. The tower of covers approach may be again deployed (e.g., applying covers of increasing resolutions as show in FIGS. 5 and 6) upon the subset embedding until an additional branch point is detected. The process is repeatedly applied until the terminal subsets of data meet a threshold. Examples of thresholds can be minimum group size, entropy or variance of the resulting subset, or other methodology that creates a terminal stopping point.

FIG. 8 depicts graph 508 with two distinct subsets of the embedded data 802 and 804. Each subset is individually re-embedded utilizing a metric, filter, or a combination of a metric and a filter.

The process repeats itself to identify new branch points for each distinct subset. In this example, the process discussed with respect to FIGS. 4A, 4B, 5-7 repeats for each distinct subset of embedded data until a new branch point is identified and new distinct subsets are created. The process can repeat again until the threshold is reached.

For example, for each of the subsets of embedded data (e.g., embedded data 802 and 804), a range of resolutions may be used to divide the embedded data space into individual sets, centroids may be determined for sets that contain data points, secondary coverings may be identified based on the centroids, and branch points determined based on non-overlapping secondary coverings to create at subsets of embedded data. The process can continue when that particular subset of embedded data is again divided into sub-subsets of embedded data and the process can continue.

FIG. 8 further depicts an example output from the RHD method showing the branch points, and process of re-embedding of subset data and applying the “tower of covers” approach to reach the next branch point.

FIG. 9 depicts a reference frame context in some embodiments. Within the initial layer of the deep topological neural network (DTNN), highly similar features are discovered across the objects. These features relate back to a reference frame with which the features are defined. FIG. 9 depicts how a set of intensity measurements of an NMR spectra can be visualized within the total NMR spectral reference frame deriving an Explanatory Element Type 1 (EET1 908).

FIG. 9 depicts a first leaf 902 (e.g., a lowest subset of any number of elements) derived from the process of the tower of covers discussed herein) of the feature space RHD 900 (e.g., the first connected-component network). The first leaf feature embedding space 904 depicts the embedded space that corresponds to the first leaf 902. The first leaf feature embedding space 904 includes isolated feature subsets 1A-1N. In this example, the EET1 908 is a section of the spectra that corresponds to the feature subset of the first leaf 902 (e.g., the terminal subset at the lowest level).

FIG. 10 depicts a second layer of the network (e.g., DTNN) in some embodiments. The isolated feature sets of high similarity (defined through a RHD using any general metric/filter combination) can be locally transposed to create a data array of objects.

In FIG. 10, similar to FIG. 9, the feature space RHD 900 includes first leaf 902. The leaf node feature embedding space 1002 depicts isolated features subsets F1A-F1N of that first leaf 902. The local transpose module 312 transposes the segment features of the leaf node feature embedding space with related objects. A sample table 1004 depicting objects 1-N as rows and isolated features F1A-F1N as columns is depicted in FIG. 10. The local object embedding space using transposed local features is depicted in graph 1006.

Here, the isolated features become the columns and the objects become the rows. A subsequent embedding of the data array illustrates distinct groupings and embedding positions. The local object space is distinct in that it can create a highly localized similarity estimation of the local features (e.g., the local features only).

FIG. 11 depicts the optional creation of metafeatures to explain segmentation in some embodiments. FIG. 11 depicts the feature space RHD 900 and leaves (e.g., leaf 902) as well as the local object embedding space of transposed local features for each leaf (e.g., leaf 902 corresponds to the local object embedding space of transposed local features 1102 for that particular leaf).

In addition to embedding coordinates, the local object embedding space may be further processed to create metafeatures that explain and describe segmentation, anomaly/outlier, and/or local hierarchy of the embedding distributions. Here, the RHD method described herein is utilized to identify unique groups within the local object space embedding (e.g., the RHD identified groups with the local object embedding space 1104). The RHD identified groups with the local object embedding space 1104 includes clusters 0-4 (e.g., the EET2 1106, which is the explanatory element type 2, local object group membership).

FIG. 12 depicts the explanation element showing group membership for a local feature space in some embodiments. FIG. 12 includes elements from FIG. 11, including the feature space RHD 900 and leaves (e.g., leaf 902), the local object embedding space of transposed local features for each leaf, as well as the RHD identified groups with the local object embedding space 1104 includes clusters 0-4 (e.g., the EET2 1106 which is the explanatory element type 2, local object group membership). FIG. 12 further depicts the explanation element showing group membership for the local feature space 1202. The line graph in FIG. 12 depicts line graphs for hybrid EET1/EET2 1204.

FIG. 13 depicts the utilization of local embedding coordinates as novel features across the local transposed elements in some embodiments. The local object space embedding components further establish features for feed-forward network propagation. Embedding coordinates (e.g., x, y, and z coordinates) of each feature of the local object embedding space of transposed local features 1302 are utilized across the local transposed elements to create a new data array unifies the object space understanding. In the example of FIG. 13, the embedding features are shown for the three components of the embedding. Feed-forward embedding features can include any number of individual components from any metric/filter similarity understanding.

The local object embedding space of transposed local features 1302 includes groups of object embedding features E1ₓ, E1_{y}, and E1z (the coordinates of E1).

Although coordinates x, y, and z are shown by example in FIG. 13, it will be appreciated that any coordinate system may be used. Similarly, although a three-coordinate system is depicted in FIG. 13, any number of dimensions may be used for coordinates to be included in the local transposed elements to create a new data array for unification of the object space understanding.

The table 1304 depicts the rows of objects 1-N with additional features (e.g., columns) including the coordinates of each feature for that related objects.

FIG. 14 depicts local clustering group information that is encoded as membership to hierarchically defined groups in some embodiments. FIG. 14 depicts local clustering group information that is encoded as membership to hierarchically defined groups in some embodiments. Graph 1402 depicts groups (i.e., hierarchically groups) that are hierarchically grouped metafeatures. In this example, graph 1402 includes grouped circular items that are associated as MF1, root triangular objects associated as MF2, grouped star objects associated as MF3 grouped diamond-shaped objects associated as MF4, and grouped square items that are associated as MF5. Explainable element metafeatures (e.g., MF1-5) are then added to table 1304 which depicts the rows of objects 1-N with additional features (e.g., columns) including the coordinates of each feature and explainable element metafeatures.

Insights and explainable elements can be further appended to the data array (e.g., table 1304) that captures embedding features for feed-forward modeling. FIG. 14 depicts that clustering group information is encoded as membership to the hierarchically defined groups (MF1, MF2, MF3). In addition, overall membership of the locally transposed group metafeatures can be annotated.

FIG. 15 depicts creation of an RHD summary in some embodiments. The summary of the RHD process can be created at the top-node embedding level of the RHD 1504 (e.g., the top node embedding of global object space RHD 1504). In some embodiments, a maximal spanning tree is created from the individual leaf-nodes of the global object space RHD. The distances and connectivity of the maximal spanning tree may be subsequently applied to the individual objects within the top-node embedding deriving distinct class understanding.

In various embodiments, the explainable machine learning system 204 may generate a visualization. A visualization may include a graph, report, interactive display, or the like depicting one or more leaf and/or subset centroids determined by methods described herein.

In FIG. 15, for example, the top node embedding of global object space RHD 1504 may be expected in a visualization as shown in the figure. In various embodiments, the visualization may show the feature space RHD 900, the global object space RHD 1502, and/or are the top node embedding global object space RHD 1504.

Some embodiments described herein permit manipulation of the data from the visualization. For example, portions of the data which are deemed to be interesting from the visualization can be selected and converted into database objects, which can then be further analyzed. Some embodiments described herein permit the location of data points of interest within the visualization, so that the connection between a given visualization and the information the visualization represents may be readily understood.

FIG. 16 depicts leaf node centroids of global object space RHD 1604 in some embodiments. For each fully connected leaf node group, the subset of data can be represented by placing a centroid at the relevant position. Centroids in the global object space RHD 1604 are depicted as large or circular objects, such as centroids 1606, 1608, and 1610 (as associated with centroids of the global object space RHD 1602).

The centroid may be calculated in a manner described by other centroids herein or in any number of ways. Size of the node (e.g., that represents the centroid) may, in some embodiments, may represent group size of the subset (not shown here).

In some embodiments, the global object space RHD 1602 (e.g., including the leaf centroids) and/or leaf node centroids of global object space RHD 1604 may be depicted in the visualization.

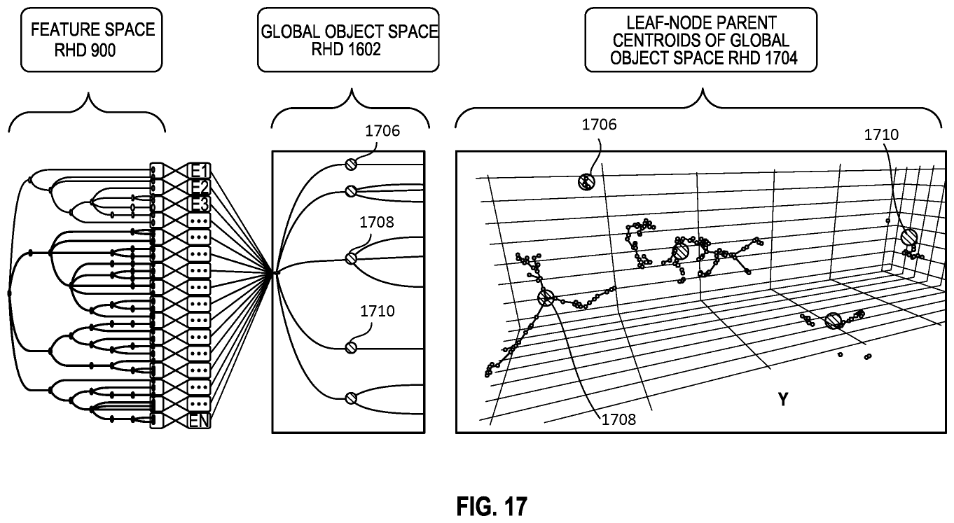

FIG. 17 depicts subset node centroids of global object space RHD 1704 in some embodiments. Working up the RHD, a centroid can be overlaid within the embedding space that approximates the relevant centroid position for each subset of data contained within the local branch. Centroids in the global object space RHD 1704 (which may correspond to the global object space RHD 1604) are depicted as large or circular objects, such as centroids 1706, 1708, and 1710 (as associated with centroids of the global object space RHD 1602).

Similar to the centroids depicted in FIG. 16, the centroids may be calculated in a manner described by other centroids herein or in any number of ways. Size of the node (e.g., that represents the centroid) may, in some embodiments, may represent group size of the subset.

In some embodiments, the global object space RHD 1602 (e.g., including the subset centroids) and/or subset node centroids of global object space RHD 1704 may be depicted in the visualization. In various embodiments, both the leaf node centroids depicted in the global object space RHD 1602 and the subset node centroids depicted in the global object space RHD 1704 may be depicted in the visualization.

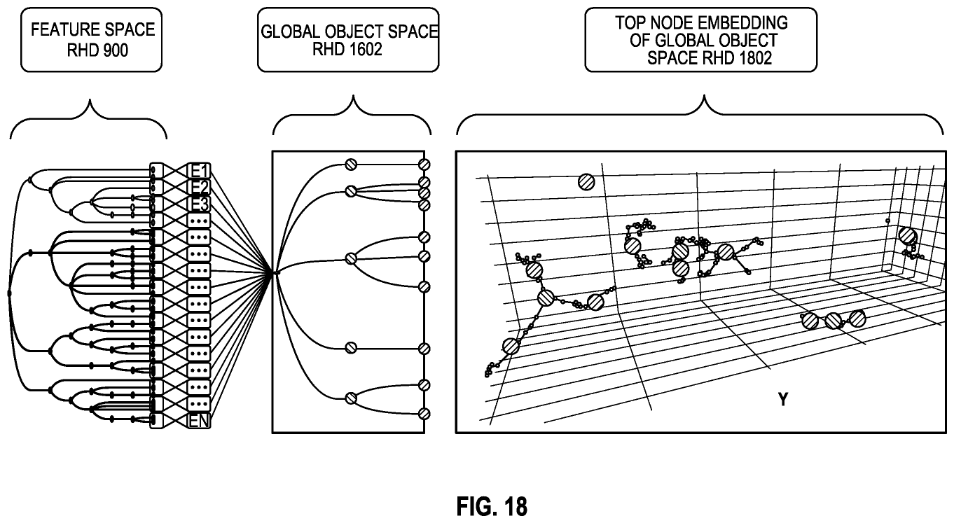

FIG. 18 depicts leaf and subset centroid placements within the embedding for each layer of the RHD in some embodiments. In FIG. 18, leaf and subset centroids are differentiated by illustrated texture (lines in different directions) and depicted in the global object space RHD 1604.

In some embodiments, the global object space RHD 1602 (e.g., including the subset centroids and leaf centroids) and/or centroids of the top node embedding of global object space RHD 1802 may be depicted in the visualization.

FIG. 19 depicts a topological summary of global object space RHD 1902 in some embodiments. The topological hierarchical decomposition shows a topological summary illustrating fully connected graph network. In this example, individual branches of the RHD summary are connected via their nearest distance of the underlying leaf nodes.

In some embodiments, the topological summary is complete when all underlying leaf node centroids are connected. Leaf nodes of the same branch node may be connected to each other and the first branch node to which it belongs. In various embodiments, leaf nodes may be connected based on a comparison of a distance metric between two or more objects or centroids of a different leaf node.

In some embodiments, the global object space RHD 1602 (e.g., including the subset centroids and leaf centroids) and/or centroids of the topological summary of global object space RHD 1902 may be depicted in the visualization.

FIG. 20 depicts an interactive visualization of the topological summary in some embodiments. In various embodiments, the topological summary can be interactively inspected. In some embodiments, Selection of a node from the summary can call the associated global object space leaf node.

The interactive visualization allows the user to observe and explore relationships in the data. In various embodiments, the interactive visualization allows the user to select nodes from the visualization. The user may then access the underlying data of the selected node (e.g., the centroid) and/or perform further analysis (e.g., statistical analysis) on the underlying data or on data as grouped within the global object space (e.g., global object space RHD selected group 2002).

In various embodiments, the user may interact with the interactive visualization depicting the topological summary of global object space RHD 1902 by selecting a centroid. In response to the selection, the interactive visualization may display the global object space RHD selected group 2002 which includes the subset of data identified by the methods discussed herein (e.g., the data for the selected centroid associated with the similar centroid of the global object space RHD 1602). It will be appreciated that the user may select any number of centroids to obtain additional diagrams graphs with the like. In various embodiments the user may be able to select one or more points or edges depicted in the global object space RHD selected group (e.g., global object space RHD select group 2002) to access the underlying data (e.g., the data from the underlying tables).

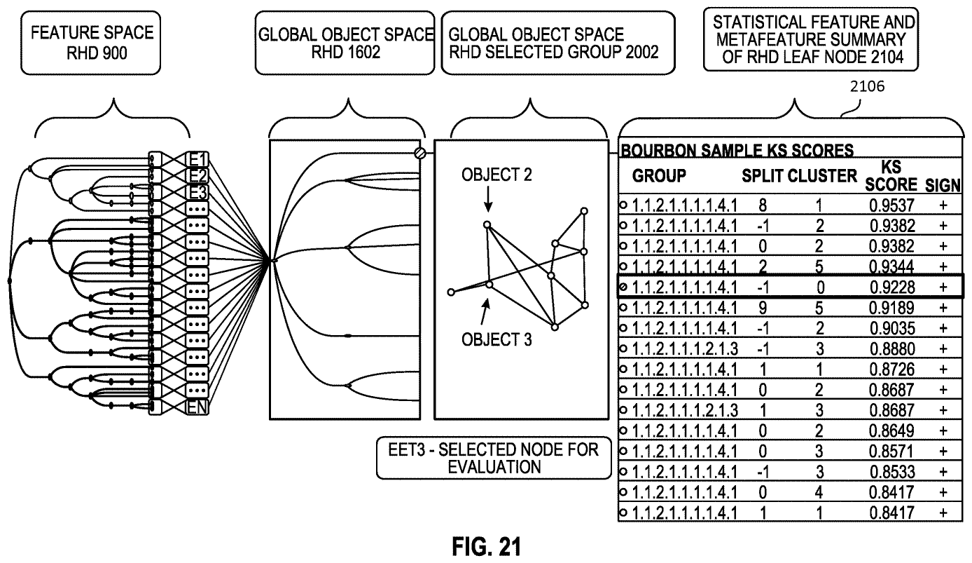

FIG. 21 depicts a statistical feature and metafeature summary of the RHD leaf node in some embodiments. The object space RHD may autonomously subset the data into individual groups through the recursive RHD process. The group subset at each node within the RHD can be statistically analyzed to understand unique features that induce segmentation. Object 2 and 3, which shared a common metafeature group assignment from a local object space model also segment together within the global object space RHD 1602. The group as a whole can be analyzed statistically to identify unique features of the RHD isolated group (e.g., within the statistical feature and metafeature summary of RHD leaf node 2104).

In the interactive visualization, a user may make a selection within the interactive visualization to depict the statistical feature and metafeature summary of RHD leaf node 2104 (e.g., table of visualization 2106). In this example, the statistical analysis includes bourbon sample KS scores. The specific feature space group can be selected for explanation visualization.

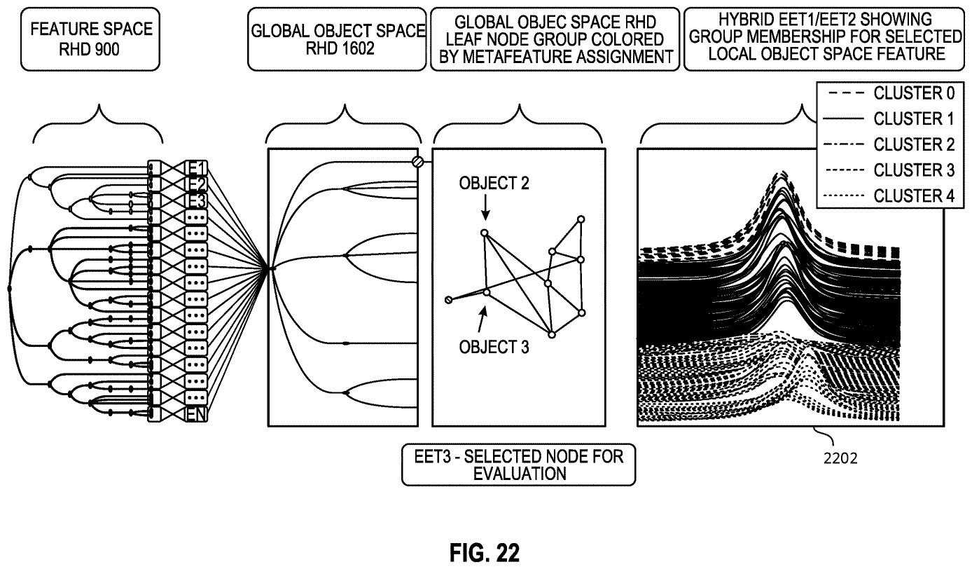

FIG. 22 depicts the hybrid EET1/EET2 showing group membership for selected local object space feature(s) in some embodiments. A global object space RHD leaf node can be colored by the underlying selected statistical features. FIG. 2202 depicts the hybrid EET1/EET2 local object space group membership as a defining feature for the selected group. Similarly, the global object space RHD can be colored by membership of metafeature group cluster assignment (blue=member of cluster 0, red=not a member of cluster 0).

In various embodiments, the visualization module 316 and/or the communication module 302 may track all transformations, and beddings, data, centroids, visualizations, and or the like and save the information a longer audit file. It will be appreciated that each step of the process from receiving of data, generating any of the connected-component networks, to projections/embeddings, identification of centroids, identification of branch points, identification of meta-features, data array creation, and/or the like can be tracked and stored for further explain ability and audit-ability. In various embodiments, a user (e.g., from a user device) may perform analysis and review the audit regarding the process for identifying inherent relationships, explanations, and the like. These audits may be useful to confirm steps, add clarity, identify areas of improvement or error, and strengthen acceptance of any conclusions.

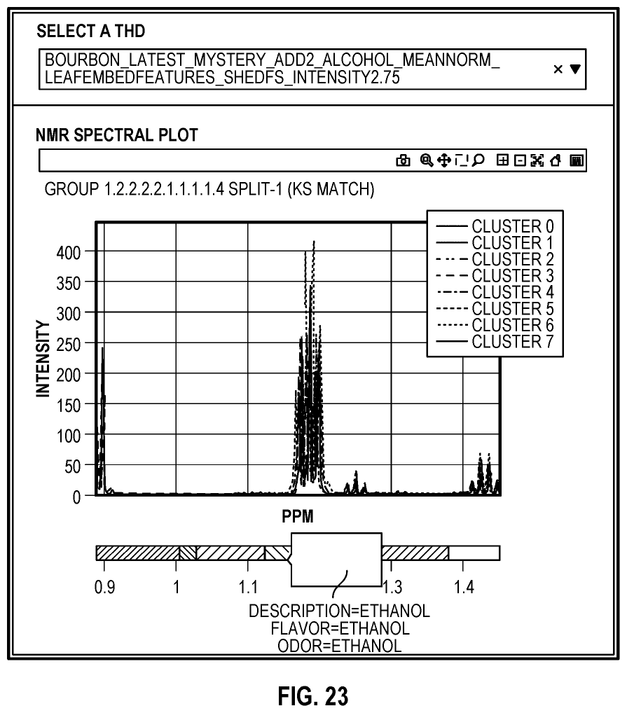

FIG. 23 depicts an interactive visualization in some embodiments. FIG. 23 depicts an example graphical user interface (GUI) for exploring unique feature sets that distinguish bourbons (e.g., using methods and/or systems described herein).

In one example of a process using methods and systems described herein is applied to bourbon analysis (e.g., analysis of bourbon). In prior analysis (unrelated to systems discussed herein) based on flavor tests, wheat bourbon's have been determined to beat rye bourbons, 12 month stave seasoning beats 6-month stave seasoning, coarse grain is preferred over average/tight, hundred 25 entry proof beats 105 entered proof, ripped warehouse beats concrete, bottom half of tree beats top half of the tree, harvest location be beats harvest location A, and char number four char number three. Barrel #80 was identified as the most preferred which was a ride bourbon, 125 entry proof, concrete warehouse, number four char, seasoned 12 month staves, bottom half of tree, and low rings per inch. In the prior analysis however, there are huge variations across customer reviews, sensory profiles, and customer preferences and general (even in expert panels).

In this example, the methodologies described herein may be applied to:
develop analytical chemistry machine learning pipelines that can develop and exploit novel patterns within the data,
develop sensory analysis methods that provide proper normalization segmentation and conductivity of metadata features across data sets, and
create highly integrated approach that enables deeper and faster identification of complex interactions that influence bourbon taste and customer preference.

The method outlined in FIG. 1 may be applied to the 80+ chemical compounds that are detected, quantified, and correlated with SOP variables and customer reviews. The data may be derived in part from gas chromatograph analysis of different bourbons. Gas chromatograph data shows that SOP bourbons largely stratified based on five variables and to a lesser extent across experimental variables. The five variables include recipe, installation date—stave seasoning, entry proof, entry weight, and harvest location. AI approaches link customer scoring and chemical composition both globally (e.g., using chemical compounds) and at the individual chemical compound level. In this example, hundred and nine SOP barrels are included in the analysis.

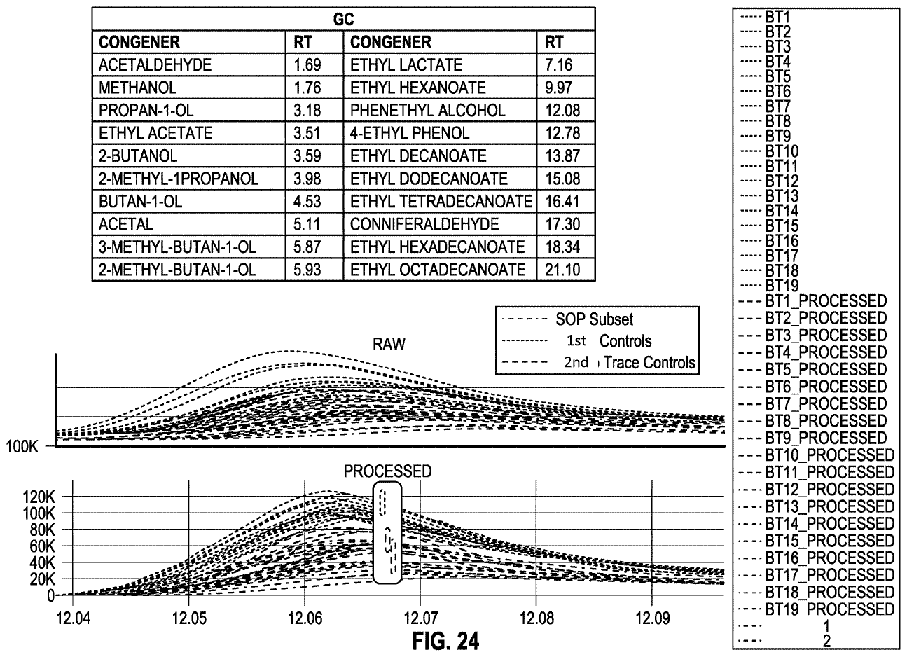

FIG. 24 is an example of a feature description for bourbons that is derived from the feature space RHD. Here, a specific range of the feature space is isolated. The user can interrogate specific traces pre- and post normalization and embeddings that may be colored by specific meta-features.

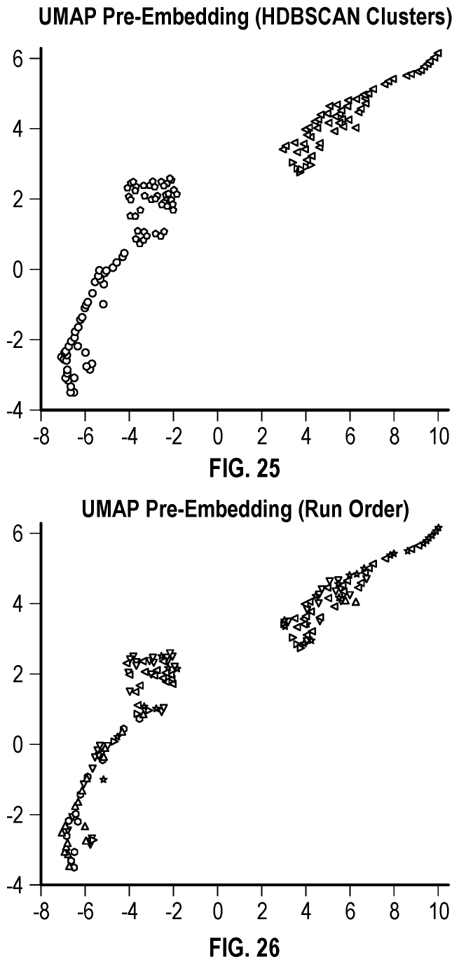

FIG. 25 depicts an example UMAP pre-embedding (HDBSCAN clusters).

FIG. 26 depicts an example UMAP pre-embedding (run order).

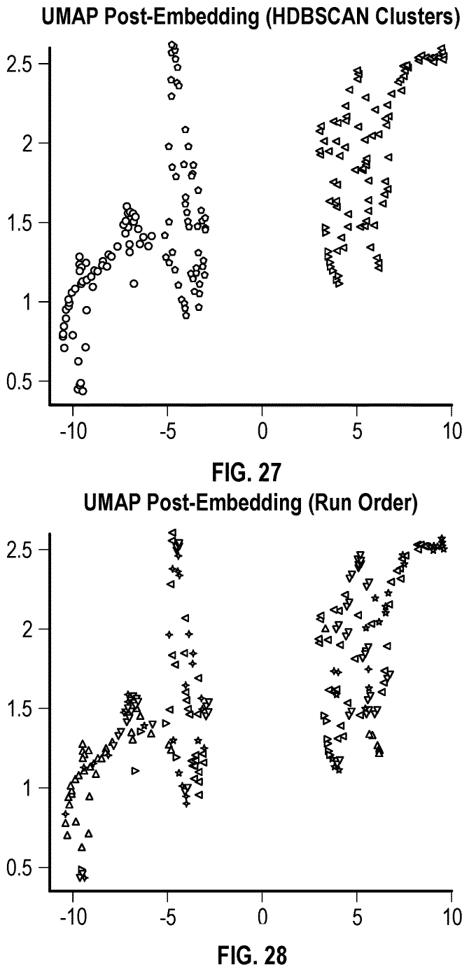

FIG. 27 depicts an example UMAP post-embedding (HDBSCAN clusters).

FIG. 28 depicts an example UMAP post-embedding (run order).

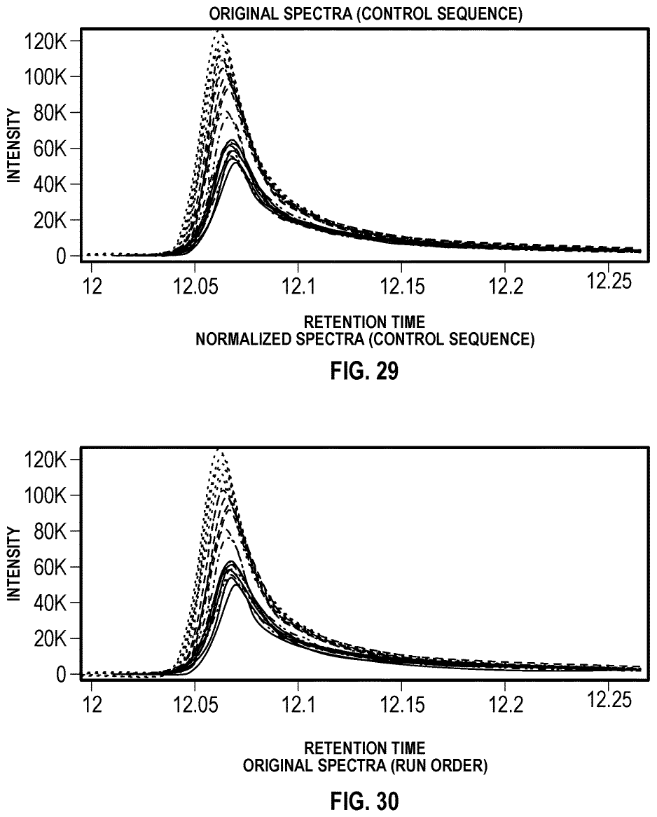

FIG. 29 depicts an example original spectra (control sequence). In this example, intensity is along the y-axis and retention time normalized spectra is along the x-axis.

FIG. 30 depicts an example retention time normalized spectra (run order) along the x-axis and intensity along the y-axis.

FIG. 31 depicts an example retention time normalized spectra (run order) along the x-axis and intensity along the y-axis.

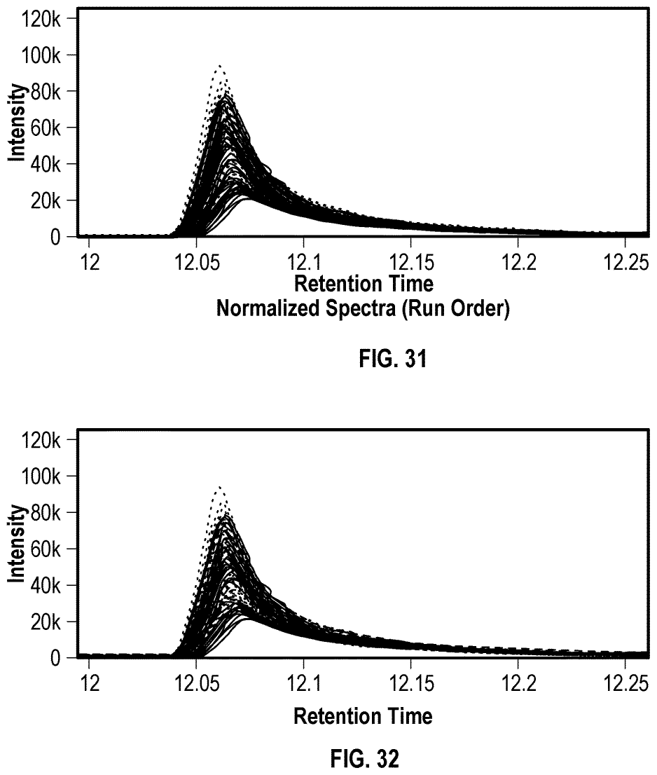

FIG. 32 depicts an example retention time along the x-axis and intensity along the y-axis.

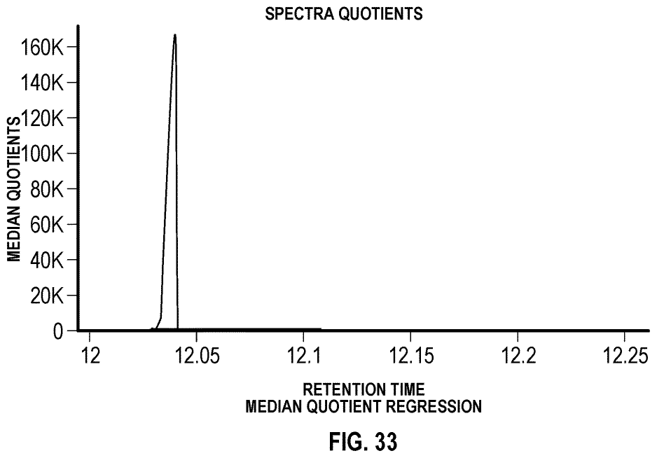

FIG. 33 depicts example spectra quotients (e.g., from the gas chromatograph). Median quotients are along the y-axis and the retention time (median quotient regression) is along the x-axis.

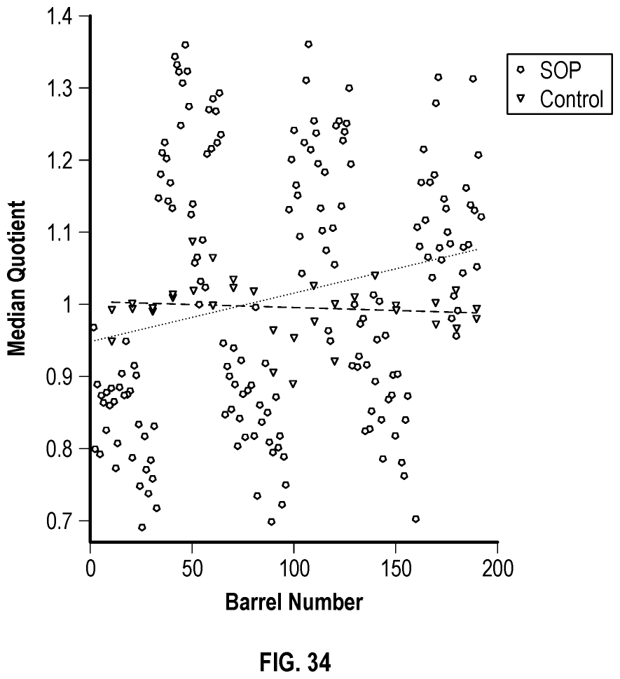

FIG. 34 depicts an example graph of SOP and control with the median quotient along the y-axis and the barrel number (e.g., for the specific barrel of bourbon) along the x-axis.

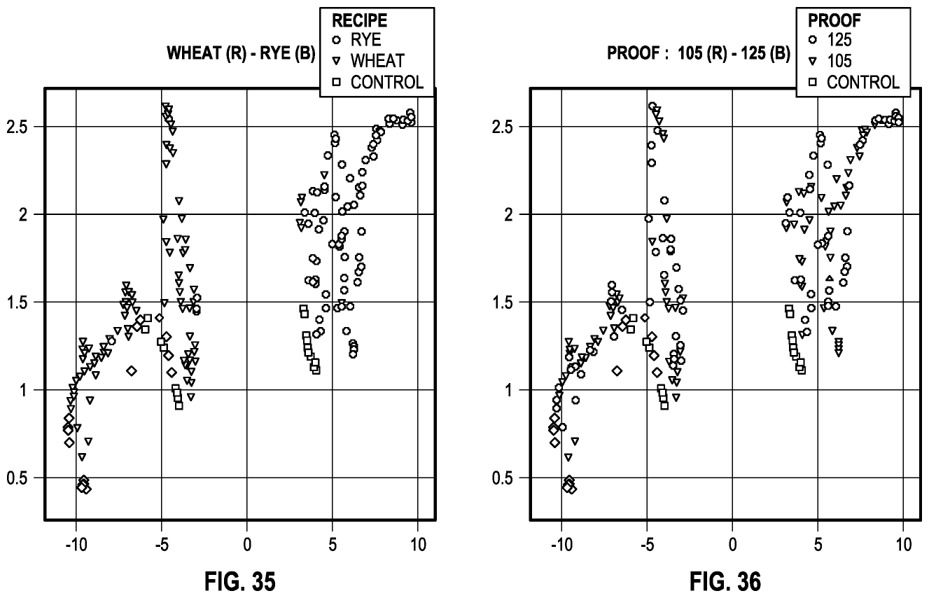

FIG. 35 depicts an example wheat to rye graph.

FIG. 36 depicts a proof 105 to proof 125 graph.

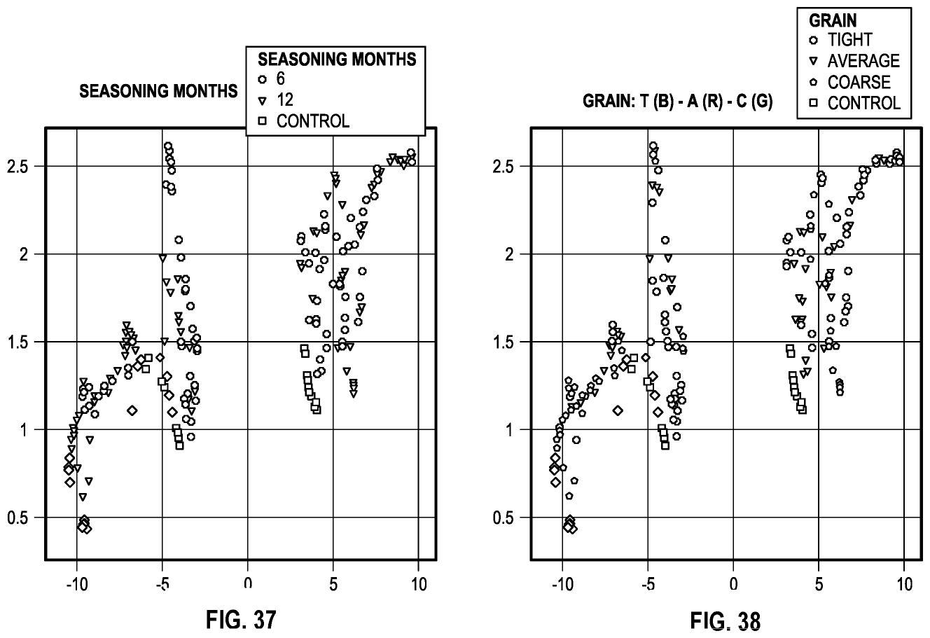

FIG. 37 depicts an example graph for seasoning of staves (e.g., comparison of 6 to 12 months).

FIG. 38 depicts an example graph of grain comparison (e.g., tight, average, coarse, and control).

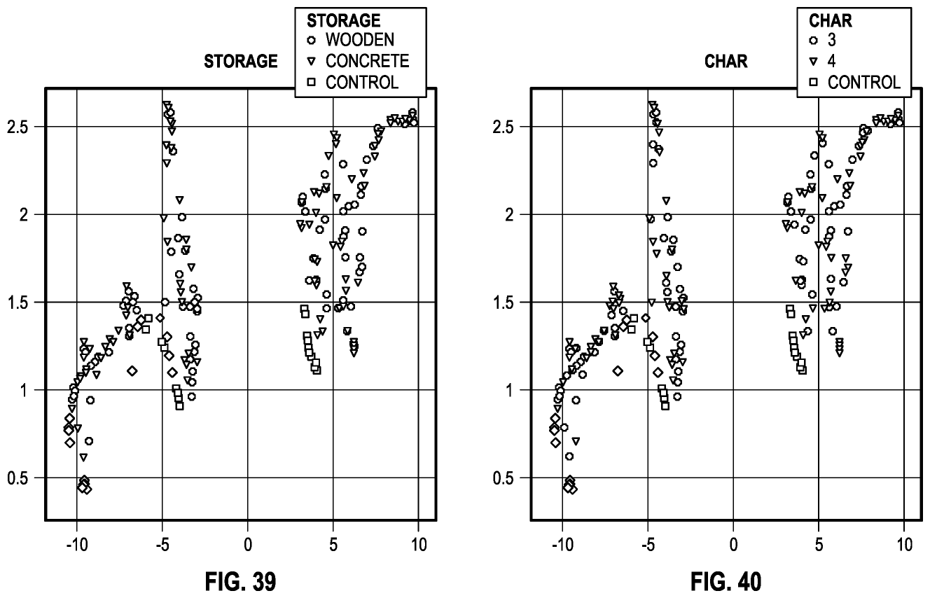

FIG. 39 depicts an example storage graph for comparison of wooden to concrete).

FIG. 40 depicts an example char graph for comparing type #3 char to type #4 char.

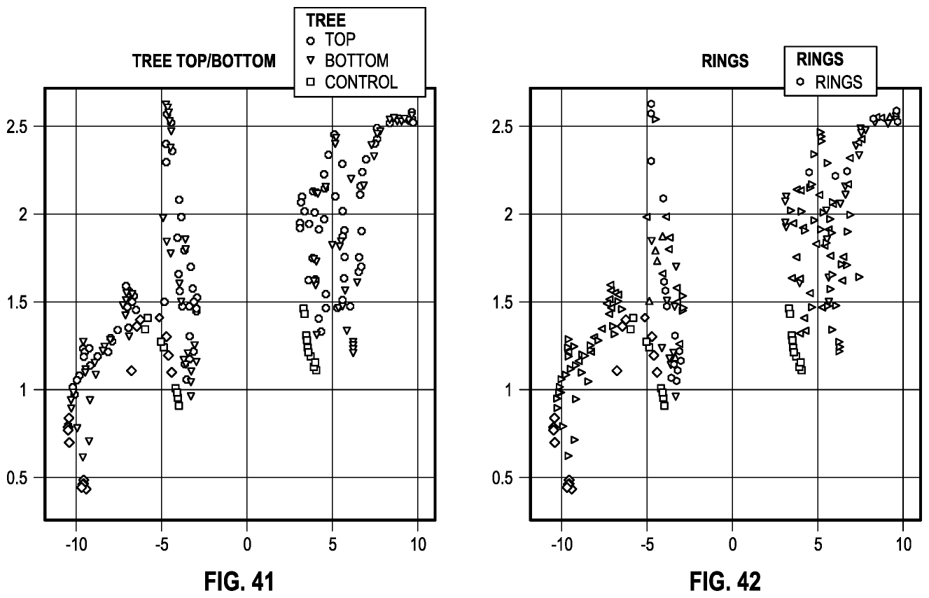

FIG. 41 depicts an example tree graph for comparison of top of tree to bottom of tree.

FIG. 42 depicts an example ring graph for comparison of the number of rings.

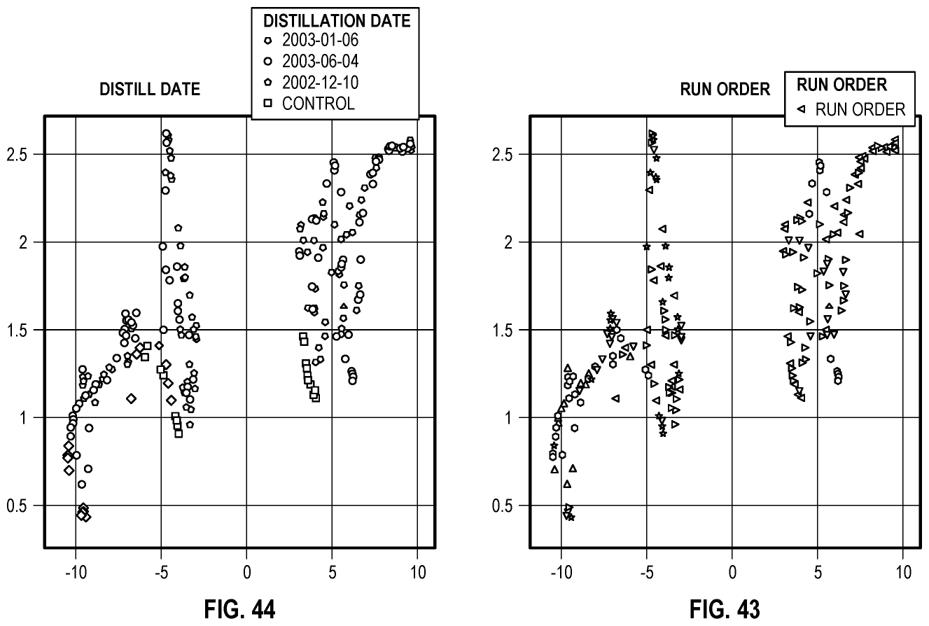

FIG. 43 depicts an example distill graph for comparison of different distillation dates.

FIG. 44 depicts a run order graph for a comparison of different run orders.

FIG. 45 depicts a block diagram of an example digital device 4500 according to some embodiments. The digital device 4500 is shown in the form of a general-purpose computing device. The digital device 4500 includes at least one processor 4502, RAM 4504, communication interface 4506, input/output device 4508, storage 4510, and a system bus 4512 that couples various system components including storage 4510 to the at least one processor 4502. A system, such as a computing system, may be or include one or more of the digital device 4500.

System bus 4512 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

The digital device 4500 typically includes a variety of computer system readable media, such as computer system readable storage media. Such media may be any available media that is accessible by any of the systems described herein and it includes both volatile and nonvolatile media, removable and non-removable media.

In some embodiments, the at least one processor 4502 is configured to execute executable instructions (for example, programs). In some embodiments, the at least one processor 4502 comprises circuitry or any processor capable of processing the executable instructions.

In some embodiments, RAM 4504 stores programs and/or data. In various embodiments, working data is stored within RAM 4504. The data within RAM 4504 may be cleared or ultimately transferred to storage 4510, such as prior to reset and/or powering down the digital device 4500.

In some embodiments, the digital device 4500 is coupled to a network, such as the communication network 112, via communication interface 4506.

In some embodiments, input/output device 4508 is any device that inputs data (for example, mouse, keyboard, stylus, sensors, etc.) or outputs data (for example, speaker, display, virtual reality headset).

In some embodiments, storage 4510 can include computer system readable media in the form of non-volatile memory, such as read only memory (ROM), programmable read only memory (PROM), solid-state drives (SSD), flash memory, and/or cache memory. Storage 4510 may further include other removable/non-removable, volatile/non-volatile computer system storage media. By way of example only, storage 4510 can be provided for reading from and writing to a non-removable, non-volatile magnetic media. The storage 4510 may include a non-transitory computer-readable medium, or multiple non-transitory computer-readable media, which stores programs or applications for performing functions such as those described herein. Although not shown, a magnetic disk drive for reading from and writing to a removable, non-volatile magnetic disk (for example, a “floppy disk”), and an optical disk drive for reading from or writing to a removable, non-volatile optical disk such as a CDROM, DVD-ROM or other optical media can be provided. In such instances, each can be connected to system bus 4512 by one or more data media interfaces. As will be further depicted and described below, storage 4510 may include at least one program product having a set (for example, at least one) of program modules that are configured to carry out the functions of embodiments of the invention. In some embodiments, RAM 4504 is found within storage 4510.

Programs/utilities, having a set (at least one) of program modules, such as the computer vision pipeline system 104, may be stored in storage 4510 by way of example, and not limitation, as well as an operating system, one or more application programs, other program modules, and program data. Each of the operating system, one or more application programs, other program modules, and program data or some combination thereof, may include an implementation of a networking environment. Program modules generally carry out the functions and/or methodologies of embodiments of the invention as described herein.

It should be understood that although not shown, other hardware and/or software components could be used in conjunction with the digital device 4500. Examples include, but are not limited to microcode, device drivers, redundant processing units, and external disk drive arrays, RAID systems, tape drives, and data archival storage systems, etc.

In the domain of artificial intelligence, a host of technologies have emerged to facilitate generative, reinforcement, predictive, and classification learning operations that can be truly transformative. However, adoption of these technologies is often constrained due to practical challenges such as small sizes of available datasets and limited model interpretability. In the field of chemometrics in particular, datasets are often constrained in the numbers of both samples and features due to significant time and labor costs required to perform precise quantitative analysis.

In some embodiments, it is possible to leverage small chemometrics datasets effectively to extract significant insights within an underlying explainable artificial intelligence (XAI) model. Some examples contained herein apply some of these embodiments on a notable experiment in the American bourbon industry called the Single Oak Project. In these examples, multiple data sources are integrated utilizing a combination of manifold learning and hierarchical clustering approaches that include customer ratings, sensory descriptors scraped from online reviews, production data, and analytical chemistry data to provide a comprehensive multi-modal understanding of how consumer preferences map empirically onto production processes, chemical composition, and sensory attributes. This XAI workflow allows linking customer sensory information to chemical composition which offers a quantitative approach to understanding individual, demographic, and population-level preferences in the American bourbon market.

Advanced artificial intelligence (AI) models are often restricted in their applicability to scientific and other domains due their dependence on large amounts of input data, their tendency to overfit results, their lack of interpretability, and a consequent distrust shared among researchers and practitioners in the soundness of results produced by such models. In the context of complex mixtures analysis, machine learning (IL) models such as Principal Component Analysis (PCA), Linear Discriminant Analysis (LDA), Orthogonal Partial Least Squares Discriminant Analysis (OPLS-DA) are among those most often employed.1-3 These simple models offer considerable interpretability, but often come at the cost of poor model performance resulting from assumptions made of the input data, such as linearity of feature relationships or linear separability of classes. In practice, these assumptions are difficult to guarantee.

The trade-offs between different AI and ML models present significant challenges to researchers performing complex mixtures analysis. Challenges arise not only in understanding the chemical behaviors observed in the collected data itself, but also in understanding the potentially complex interactions between the variables that characterize the physical and chemical processes which the samples bear evidence to. As dataset sizes are often relatively small when conducting analytical chemistry research (e.g., experiments frequently involving dozens or hundreds of observations at a time) models requiring large amounts of training data are frequently unsuitable for use. Additionally, since model interpretability is vital in a scientific context, the models employed in complex mixtures studies often rely on some form of dimensionality reduction, typically via PCA or similar linear methods. However, assumptions of linearity in the input data along with the coarse resolution of detected structure can lead to such models failing to produce meaningful results, even when significant global relationships are present between classes in the observation set.

In some embodiments, an explainable artificial intelligence (XAI) architecture employs topological hierarchical decomposition (THD) as its principal analytical unit. Since THD models may, in some embodiments, operate exclusively from low-dimensional representations of the input data, they may be well-suited for use in domains where model interpretability is a priority.

In various embodiments, THDs discussed herein are extended to include a “local transform” operation for THD feature analysis. Some embodiments further include a general method for performing multimodal data fusion within the context of THD analysis, offering multiple approaches to characterizing the learned relationships between sets of input features and observations spanning multiple data modalities in explainable summary models. Examples for one or more of these approaches and operations are discussed herein with reference to an analysis of the Single Oak Project, a landmark experiment in the distilled spirits industry conducted at Buffalo Trace Distillery in Frankfort, Kentucky.

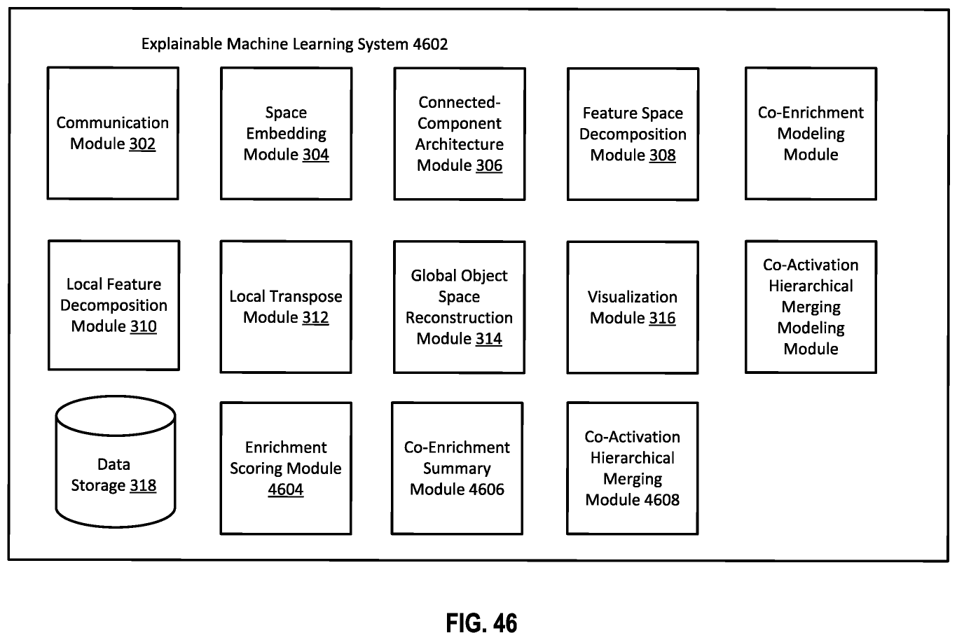

Various embodiments utilize the explainable machine learning system 204. FIG. 46 depicts an explainable machine learning system 4602 in some embodiments. Some embodiments utilize the explainable machine learning system 4602 which may include similar components to the explainable machine learning system 204, including the communication module 302, the space embedding module 304, the connected-component network module 306, the feature space decomposition module 308, the local feature decomposition module 310, the local transpose module 312, the global object space reconstruction module 314, the visualization module 316, the data storage 318, as well as the enrichment scoring module 4604, the co-enrichment summary module 4606, and the co-activation hierarchical merging module 4608 discussed herein. In some embodiments, the explainable machine learning system 4602 includes the enrichment scoring module 4604 and the co-enrichment summary module 4606 but not the co-activation hierarchical merging module 4608. Alternately, in other embodiments, the explainable machine learning system 4602 includes the co-activation hierarchical merging module 4608 but not the enrichment scoring module 4604 or the co-enrichment summary module 4606.

In one example discussed herein, gas chromatography is used with flame ionization detection (GC-FID) in complex mixtures analysis. The most common approaches to GC-FID analysis aim to determine exact concentrations of target analytes, requiring both the careful identification of target peaks in the chromatographic data as well as the calculation of peak response calibration curves for each analyte. These prerequisite steps demand a non-trivial contribution of time and labor before precise quantitation work can begin, restricting the number of analytes that can be used to study real-world processes as well as the throughput of labs performing GC-FID analysis. Furthermore, such restrictions on throughput limit the viability of AI/ML models which require large amounts of training data. While several methodologies have been proposed which seek to automate chromatographic peak detection and concentration analysis, these methodologies often struggle to deliver results that are accurate, reproducible, and timely.

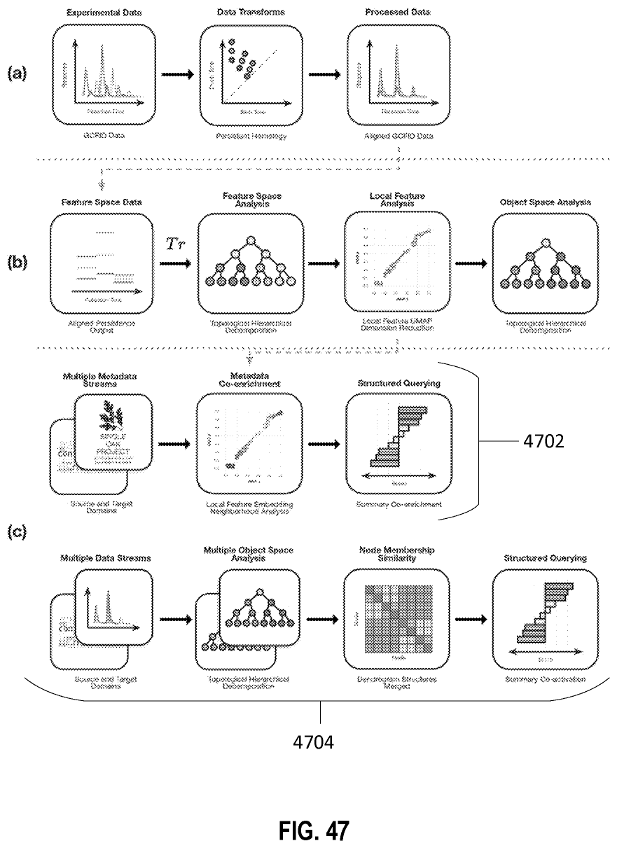

The example workflows described with regard to FIGS. 47A-C do not rely on absolute concentration measurements. Instead, these example workflows make use of relative responses measured with respect to an internal chemical standard of known concentration added to each sample, dispelling the need for analyte identification and calibration, and thereby expanding the set of usable peaks for statistical analysis. This approach may be sufficient for most experimental work involving the analysis of independent and dependent variables or multimodal data sources external to the sample chemistry, as well as the determination of analyte significance in relation to these variables. In some embodiments, these workflow utilize not only the multivariate and potentially nonlinear nature of the chemistry of complex mixtures, but also that of external phenomena such as sensory perception, which might arise from complex, nonlinear interactions between several molecules rather than the mere abundance or absence of individual compounds.

Hierarchical clustering using THD is utilized in some embodiments. As discussed herein, THD is a divisive hierarchical clustering algorithm that utilizes iterative manifold learning to produce an encoding of multi-scale topological structure within a dataset. The use of low-dimensional manifold representations may ensure interpretability of the resulting model and may drastically improve the clusterability of the data. Performing manifold on each data cluster may further improve both global and local structure preservation for subsequent sub-clusterings. In one example, Uniform Manifold Approximation and Projection (UMAP) is utilized for manifold learning within THDs, as it offers a reasonably strong balance of both local and global structure preservation as well as fast computational performance, particularly in its GPU-accelerated implementation.

Hierarchical clustering methods have a long-standing history of use throughout machine learning, however, little emphasis has been placed on using hierarchical clusterings to merge information from multiple sources of input data. Most machine learning methods which make use of multiple hierarchical representations of an observation set typically do so with respect to a single set of input features while varying, for example, the parameters governing dendrogram construction.

Some embodiments described herein assist to overcome these limitations by utilizing enrichment scoring functions (defined herein) as a means of quantifying simultaneous localization of information from multiple input features, either within or across feature modalities. A measure of nonlinear correlation may be based on enrichment scores and may be used to construct summary topological models of feature relationships. Additionally or alternatively, some embodiments described herein utilize a methodology by which information from two hierarchical clusterings is compared and subsequently merged to yield a single hierarchical model which summarizes the information and data subsets common to each of the input models. The summary models produced by these two example methodologies may offer significant advantages in their abilities to elucidate complex interactions between features spanning multiple data modalities. As a result, each model separately or in any combination may offer a strong basis for performing robust exploratory analysis of datasets involving complex, multimodal input features. Further, the embodiments described herein offer a valuable basis for constructing more complex explainable deep learning models.

An example application included herein is an analysis of the Single Oak Project (SOP), a landmark experiment in the distilled spirits industry. This example application is an effort to understand the impact of various production decisions on the taste, color, aroma, and preference of single barrel bourbon. In this example, seven production parameters were varied to produce 192 unique barrels of bourbon which were bottled and released to the public between 2011 and 2015.23 consumer feedback was collected over the course of the release and analyzed to derive broad correlations between production variables and consumer preferences. Further, in this example, GC-FID was utilized to characterize the chemical composition of samples taken from the barrels produced for the experiment. Using various sources of metadata including production parameters, consumer sensory descriptions, and numerical consumer ratings of each barrel, the following is an example comprehensive analysis of the SOP that utilized barrel chemistry as the primary modality through which otherwise disparate sources of metadata could be authoritatively related to one other.

FIG. 47A-C depicts workflow overviews used in the analysis of the Single Oak Project in some embodiments. FIG. 47A depicts the preparation of GC-FID data for the calculation of peak prominence data and alignment of chromatograms across experimental samples.

In various embodiments, the communication module 302 receives GC-FID data.

GC-FID data may be collected in any number of ways. In one example, GC-FID data is collected as follows: In a vial 1 g of internal standard may be added to 9 g of sample. The sample with the internal standard may then poured into a 2 mL GC vial and placed onto an autosampler. An Agilent 7890B GC equipped with a J&W DB-624 ultra inert 30 m×250 mm×1.4 m column and a flame ionization detector may be used for chemical analysis. The GC inlet may be set to a 20:1 split and both the inlet and the FID were set to 250° C. The GC may be set to a constant flow of 1.25 mL/min and the oven profile was as follows: 40° C. 1 minute hold, ramp 18.5° C./min to 80° C., 2 minute hold, ramp 18.5° C./min to 100° C., ramp 22.2° C./min to 120° C., 2 minutes hold, and finally ramp 22.2° C./min to 250° C., and held for 10 minutes. A Gerstel MPS-xt autosampler may be used to inject 1 μL of sample with internal standard into GC. After every 10 samples, a QC with a known concentration of analytes and two whiskey standards were injected into the GC. All samples may be run on the GC in a single, continuous batch. Analysis may be performed using Agilent's MassHunter Workstation Software Quantitative Analysis. The rainbow-api package for Python may be used to read data from Agilent directories and to extract the FID channels. Linear interpolation may be used to a shared set of retention times across the full set of samples. For denoising, an average may be taken between two rolling window smoothing results at window sizes of 50 and 100 data points. Local polynomial regression as implemented within the pybaselines Python package may be used to fit baseline curves for removal.

In GC-FID analysis, a variety of instrumental factors may result in retention time drift, yielding inconsistencies in the retention times at which any given compound may elute within samples run over the course of an experiment. For complex mixtures with non-trivial peak structures, performing careful peak alignment without significant degradation of peak areas presents a challenge. Popular GC-FID peak alignment methodologies involve learning a warping function (e.g., correlation optimized warping, dynamic time warping) for each chromatogram. This is computationally expensive and often results in deformation of the peaks being targeted for measurement, even under constraints specially determined for GC-FID data.

In various embodiments, the communication module implements an alignment procedure that aims to preserve peak shape for peaks above a user-defined prominence threshold. It will be appreciated that, regions of a chromatogram containing no significant peak information (as determined by peak prominence) may be freely relocated without any change in peak topology, which enables shifting non-overlapping peaks as well as contiguous regions of overlapping peaks without interfering with peak area measurement. If further alignment of overlapping peak sets is required, constrained warping function methods may be used with reduced impact on peak shape, although none were used for the analysis of this experiment.

To align the peaks throughout the SOP chromatographic data, a matching between peaks may be compared by the communication module 302 using an unbalanced bipartite graph matching algorithm implemented within the NetworkX Python package. A rigid global alignment may be determined using the median error between the peak locations of peaks in each chromatogram and those in a reference sample. A flexible local alignment may be performed using the prominence-based peak alignment methodology discussed above using a prominence threshold of 0.05.

FIG. 47B depicts application of topological hierarchical decomposition to perform feature analysis, and the local transform operation on the sample set. As discussed herein, the feature space embedding module 304 may generate a lower dimensional embedding feature space by projecting the data based on metrics and/or filters discussed herein. The connected-component network module 306 may generate connected-component networks (e.g., using the “tower of covers” approach discussed herein). The process is discussed with regard to FIG. 4B.

The feature space decomposition module 308 may generate a lower dimensional embedding of the feature space as described herein for each leaf of the first connected-component network as described herein.

In this example, given a data matrix X, where observations are given by rows and features by columns, clustering operations yield sets of observations which exhibit similar distributions of values across the data features. In this example, clustering operations may be performed on the transpose of the data matrix X\hat{ }t, also called the feature space orientation. In feature space, then, cluster analysis yields sets of features which exhibit strong similarities across the observation set. In the GC-FID experimental setting, a topographic characterization of the chromatographic data is utilized as input for feature space cluster analysis using THD. The resulting clusters contained retention times which exhibited similar response patterns throughout a number of observations, typically, but not always, corresponding to individual analyte peaks. In this example, a total of 68 feature clusters are obtained after selecting only those leaf nodes of the THD which contained prominences exceeding a specified threshold.

As discussed herein, the local feature decomposition module 310 may assist in identifying features in individual leaves of the feature space for embedding in the leaf node feature embedding space or generating the local object embedding space used to transpose local features as discussed with regard to FIG. 10. The local transpose module 312 is configured to locally transpose the RHD isolated feature sets (e.g., objects as rows and RHD isolated features as columns) as discussed herein.

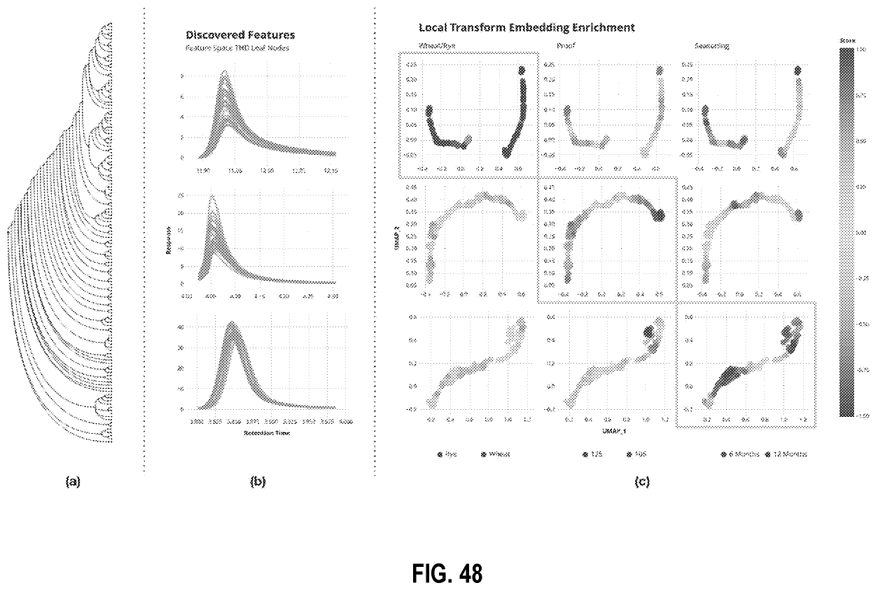

In this example, once a set of feature clusters was obtained from feature space analysis, slices of the data matrix in object space are generated by restricting the columns to those contained in each feature cluster. FIG. 48 depicts feature clusters in some embodiments.

As each subset was high-dimensional, in some embodiments UMAP may be utilized to generate local dimensionality reductions of each slice. This process of generating local feature reductions of a data matrix is referred to as the “local transform” operation herein. Prior to dimensionality reduction, area features may be generated from each slice of the chromatographic data, calculated by integrating over retention time samples selected according to a sliding Gaussian distribution over each slice. It will be appreciated that any distribution may be utilized. These sets of area features may be taken as the inputs for each local dimension reduction. The resulting embeddings contained detailed information about the distributions of analyte peak responses across the observation set. FIG. 48, section C, depicts that the embeddings frequently indicated continuous one-dimensional manifold structure. It will be appreciated that embeddings exhibiting more complex structure and stronger clustering behavior may be frequently observed.

Local transform embeddings may provide a compact feature set for representing a given data matrix and may enable interpretation of the discovered features. Section A of FIG. 48 depicts a feature space THD of persistence score data in some embodiments. Section B of FIG. 48 depicts three chemical features discovered in the feature space THD analysis, each representing a leaf node of the dendrogram in some embodiments. Section C of FIG. 48 depicts a local transform embeddings for each of the peaks shown in (b), given row-wise, colored by their enrichment scores (introduced below) in three different metadata features, given column-wise in some embodiments. Outlines are used to indicate which metadata feature each embedding exhibits the strongest enrichment in, although each embedding may localize a number of metadata features simultaneously.

In the example discussed herein, for feature space modeling, a THD may be trained using UMAP for the manifold learning steps and [clustering algorithm] for clustering each embedding. The following parameters in this example may be used for UMAP: n_neighbors=50, metric=“manhattan”, n_epochs=3000, n_components=3. A stop threshold of 100 data points may be used for the decomposition. In this example, after the THD is computed, a set of feature clusters may be determined by filtering any feature clusters that did not contain peaks above a prominence of 0.05 and by separating contiguous regions within each feature cluster separated by a retention time gap of 0.01 minutes.

For the local feature embeddings in this example, sampled areas may be taken from each THD feature cluster to use as input features for the embedding. To generate the samples, a sliding truncated Gaussian distribution with variance scaled by the width of the feature retention time interval may be used to produce 30 samples of min(30, N) retention times each, where N is the number of time points in the interval. 30 approximate area samples may be calculated using trapezoidal integration over each set of sampled retention times. The UMAP embeddings may be computed using the following parameters: n_neighbors=30, n_components=3.

To compute the object space model in this example, the embedding coordinates from the full set of local feature embeddings may be concatenated to form a single, compact feature set. The object space THD may use the following parameters for its internal UMAP embeddings: n_neighbors=10, n_epochs=2000, min_dist=0, spread=1.5. Fiedler vector clustering may be used to cluster the data at each branch point with a stop threshold set to 5 data points.

The global object space reconstruction module 314 may generate the global object space, the top node embedding of the global object space RHD 1504, and/or the topological summary of global object space RHD 1902 as described with regard to FIG. 13-19.

FIG. 4702 depicts an example method for explainable analysis in some embodiments.

As discussed herein, various embodiments of systems and methods include generation of a component-connected architecture. The component-connected architecture may enable the discovery of relationships of features within high-dimensional spaces.

As discussed similarly in FIG. 4A which depicts a method for generating explainable insights using component-connected architecture(s) in some embodiments, the communication module 302 retrieves or receives data from one or more data sources (e.g., data sources 110A-N). The data may be in any form or organization.

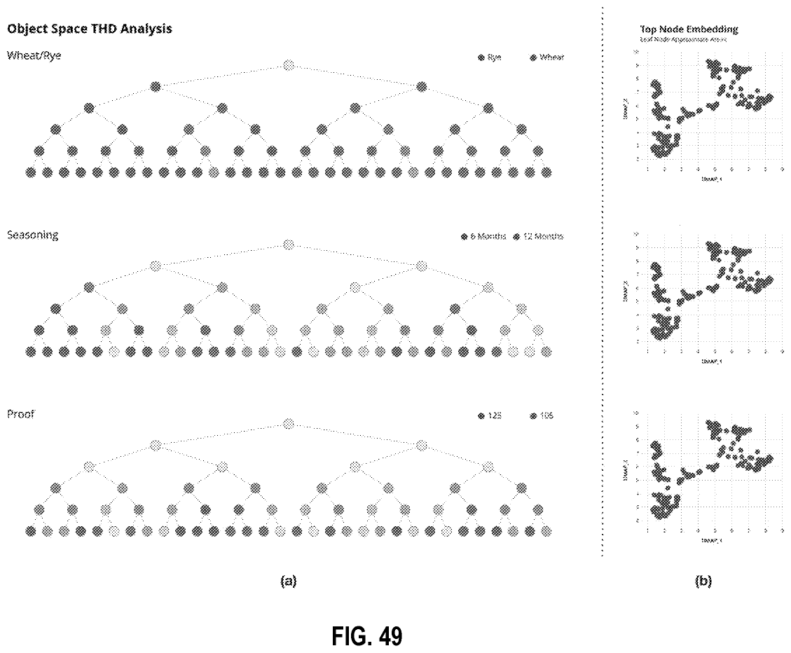

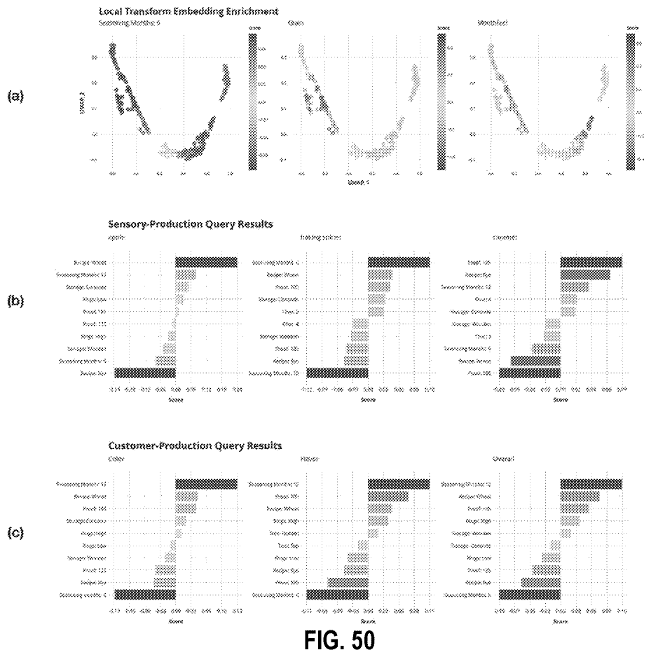

To unify the local transform information into a singular object space model, the local transform embedding coordinates generated from the feature space THD leaf nodes may be used as input features for object space THD analysis. The resulting clustering may provide an interpretable model of compositional differences between discovered classes of bourbon samples. In this example, there is a strong correspondence with the SOP production variables as shown in FIG. 49. FIG. 49 depicts an object space topological hierarchical decomposition in some embodiments. In nodes which were found to correspond to a separation of production classes, the node height within the dendrogram may be taken as a proxy for the discriminative strength of that production feature within the node's contained object subset.

In the example depicted in FIG. 49, results of object space THD analysis of the Single Oak Project GC-FID data using local transform embedding coordinates are utilized as input features. In the example depicted in FIG. 49, section (a), hierarchical clusterings of the input data given by a THD, may be colored by production metadata features. Section (b) of FIG. 49 depicts the root node UMAP embedding of the THD colored by the production metadata features corresponding to the rows of (a).

Returning to FIG. 47, section C depicts two approaches to summarizing correlative relationships between disparate metadata sources. In the first approach 4702, local transform embeddings are used to measure the extent by which metadata features are simultaneously localized by individual chemical features. These “co-enrichment” scores are then summarized to provide a measure of correlation between metadata features as a function of sample composition. In the second approach 4704, hierarchical models constructed from disparate input feature sets are merged to produce a single combined model which summarizes correlation between input data features.

In some embodiments that utilize multimodal fusion, the enrichment scoring module 4604 compares features across secondary data modalities (e.g., one source modality and one target modality) through their commensurate representations in the primary data modality. Leveraging direct sample correspondences between data modalities, simultaneous enrichment of features may in neighborhoods throughout local feature clusters may be measured using “enrichment scoring.” In some embodiments, enrichment scores measure the prevalence of a metadata feature in some target sample P relative to its prevalence in some reference distribution Q. An example enrichment scoring function is as follows:

$$ES ( P , Q ) = tanh ( β \cdot sgn ( {μ}_{P} - {μ}_{Q} ) \cdot \frac{{W}_{1} ( P , Q )}{{σ}_{Q}} )$$

where W₁ is the Wasserstein 1-distance.

In some embodiments, to calculate enrichment scores within local transform embeddings, the KNN neighborhoods of each observation may be taken as target samples and the entire observation set as a fixed reference sample. Enrichments of both the source and target features for all sample neighborhoods in all local transform embeddings may be calculated. These scores may result in a data tensor, transformations of which were may be used to assign a measure of correlation to secondary feature relationships (the total simultaneous enrichment or “co-enrichment” of the features) and to assign attribution scores to both objects and features in the primary modality.

FIG. 50 depicts examples of enrichment scores measured in a local transform embedding in some embodiments. Co-enrichment between two metadata features in an embedding may be quantified by element-wise vector multiplication of their associated neighborhood enrichment scores. The resulting vector may capture the neighborhood-wise local correlation between the pair of features within the observation set.

To derive an overall measure of correlation between pairs of secondary metadata features with respect to the primary data modality, the pairwise co-enrichment scores may be summed across all local transform embeddings and normalized the results. In some embodiments, this normalization may be designed to bound the summary scores between −1 and 1 (although any range may be used), to obtain a value of 1 only when the corresponding features have identical unital neighborhood enrichment scores in all local transform embeddings, and to obtain a value of −1 only when they exhibit exactly opposite unital enrichment scores in all embeddings. This final summary score for each feature pair may be described as a “query score.” In various embodiments, since computing a query score from two enrichment vectors amounts to computing a normalized dot product, a full matrix of query scores between pairs of metadata features can be computed via a normalized matrix multiplication operation.

It will be appreciated that the co-enrichment methodology leverages reconstructions of the object manifold using local views of the primary feature manifold to measure the local information content present in each with respect to some set of secondary feature modalities. From this, a nonlinear measure of arbitrated correlation between secondary features may be derived, as well as attribution scores for both the objects and primary features. In this example analysis of the SOP, the GC-FID data is taken a primary modality and computed query scores between the secondary production and sensory data modalities, as well as between the production and consumer preference data.

In this example, within each local feature embedding, enrichment scores for the various metadata features are computed using the 30 nearest neighbors of each point. These may be concatenated to form a matrices of shape (n_features, n_groups*n_samples) for each metadata feature set. Given two such matrices for one source and one target feature set, the source matrix may be multiplied by the transpose of the target matrix to produce a matrix of size (n_features, n_features). This final query matrix may be summarized using a UMAP model with the following parameters: n_neighbors=50, metric=“correlation”, n_epochs=2000, spread=3.

To produce feature maps back to the GC-FID local feature clusters in this example, the original matrices of enrichment scores may be reshaped to form matrices of size (n_features*n_groups, n_samples) for each metadata feature set. In this orientation, matrix multiplication of a source matrix and the transpose of a target matrix may be performed to calculate a set of query scores. The average is then taken to yield a final score for each feature.

In the example of FIG. 50, local transform embeddings may offer a convenient setting for calculating and visualizing correlative relationships between metadata features. Section (A) depicts a local transform embedding colored by KNN neighborhood enrichment in three different metadata features. Barrels seasoned for six months (left) exhibit strong positive co-enrichment with barrels that reviewers described as tasting strongly of grain (center) and exhibit strong negative co-enrichment with barrels described as having a pleasant mouthfeel (right). Note the difference in colorbar scales. Section (B) of FIG. 50 depicts query scores indicating relationships between three sensory metadata features and the production metadata feature set. Section (C) of FIG. 50 depicts query scores indicating relationships between three customer rating metadata features and the production metadata feature set.

To summarize the information contained in a matrix of query scores about feature relationships between a source and target modality, the co-enrichment summary module 4606 may first append to it those scores generated by measuring co-enrichment between the source modality features and themselves. This appended set of feature scores serves as a set of reference landmarks for contextualizing cross-modality feature relationships in the target modality in downstream summary models. In the example analysis of the SOP, the neighborhoods of the resulting data matrix contained sets of sensory and production metadata features with chemically derived correlative relationships, which may be visualized using UMAP. To automate the search for neighborhoods of chemically related features with maximal cohesion within this summary model, THD cluster analysis of the query scores data may be performed. The resulting model offers a compact and concise basis for exploring sets of features which cohere across multiple data modalities in light of a shared data representation.

An alternative approach to multimodal fusion leverages the fusion of multiple hierarchical clustering models, one source and one target, constructed using differing sets of input features defined on identical sets of objects. To illustrate this approach, the explainable machine learning system 4602 constructed an object space THD on the production metadata, which was weighted exponentially according to total feature enrichment to induce a hierarchical structure. Taking this production THD as the source model and the chemical object space THD shown in FIG. 48 as the target model, the co-activation hierarchical merging module 4608 merges two hierarchies. In this example procedure, a matrix of similarity scores is calculated, scoring the similarity of each cluster in the source THD with each cluster in the target THD in terms of shared objects. This may provide a measure of “co-activation” between the dendrograms. To this data matrix a matrix representation of the source THD dendrogram structure is appended, and a final “merge” THD may be constructed for this data matrix. The example root node UMAP embedding of this THD is shown in section B of FIG. 50, along with an analysis of one of its leaf nodes. In this example, the final merge THD model positions each cluster in the query THD in context of the target hierarchy and their shared objects, giving a semantic contextualization of the source hierarchy, and providing an interrogable and explainable model of object and feature relationships between the input data modalities which emerge in the final dendrogram.

In one example of co-activation hierarchical merging, two THDs are constructed for co-activation merging, one on the source data and one on the target data. Each node in the source THD may be then compared to each node in the target THD by calculating their overlap coefficient, a Jaccard-like scoring function:

$$f ( U , V ) = \frac{\vert U ⋂ V \vert}{min ( \vert U \vert , \vert V \vert )}$$

These scores may be used to construct an affinity matrix A, which may then be concatenated with a “group membership” matrix G of the source THD, constructed such that G_{i,j}=1 if object xᵢ is in group gⱼ, otherwise G_{i,j}=0.

A third THD may then fit on this concatenated matrix. The UMAP parameters used for the THD are as follows: n_components=2, n_neighbors=60, metric=“correlation”, spread=5. Fiedler vector clustering is used with a stop threshold of 5 data points. To construct the feature maps for a given group in the THD, the path to the group from the root node may be first obtained. Then, for each node in the path, enrichment scores in the local transform embeddings may be calculated using that node's column entry in the THD's group membership and their absolute values are averaged within each feature cluster.

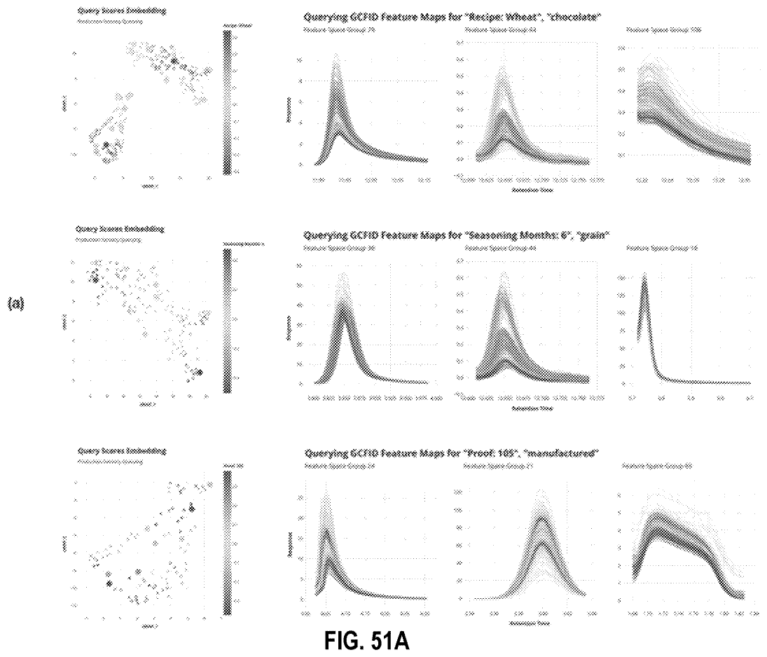

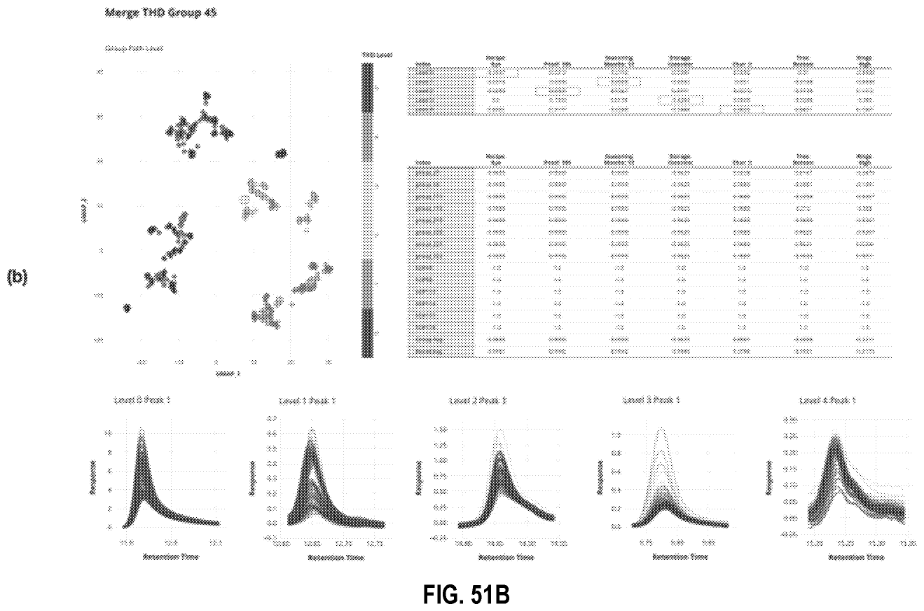

For example, in FIG. 51A, the path through the merge THD dendrogram of a leaf node containing six SOP barrels is traced. In FIG. 51A, the difference in cluster enrichment scores in the production data between child and parent nodes in this path is measured, broken out by parent depth in the dendrogram. The cells in the lower part of the graphs (e.g., blue-bordered in some embodiments) in this table show the maximum score for each depth level, giving an interpretation of which production variable most significantly accounts for the clustering given at that node. For the example cluster of six barrels shown, the objects are distinguished by 5 of the 7 production variables. Moreover, the depth at which each production variable becomes enriched serves as a proxy for how globally or locally significant the variable enrichment is. To validate that the information contained in the source THD clustering is preserved in the merged hierarchy, the middle table depicts the cluster enrichments for the source THD nodes contained in the same leaf node as the six SOP barrels. These may come from the appended matrix representation of the source THD hierarchy. The scores shown in the middle table may demonstrate that each production variable becomes statistically significant at some degree of sample localization. In this example, this held true for nearly all leaf nodes of the merge THD. In some embodiments, the co-activation multimodal fusion approach demonstrates that object space THDs can semantically feed information forward through a simple merge process.

FIG. 51A depicts an example of query scores overlaid on a metadata feature summary embedding showing which neighborhoods of metadata features pertain most directly to a given metadata feature (left). Aggregated activation scores for each observation, used to visualize the most relevant subsets of the GC-FID data for a given query (right).

FIG. 51B depicts an example co-activation embedding of chemical and production object space THD clusterings (top left), colored by depth in an “activation path” from the root node to an example leaf node of the merge THD. In this example, differential enrichment scores are shown for each node in the activation path, the difference being taken between each path node and its children (top right). Blue boxes highlight the most significant production variable associated with each node. In this example, raw enrichment scores for each observation in the example leaf node (center right). The samples in each node of the activation path (e.g., which may be in red), used to visualize object relevancies within candidate chromatographic peaks (bottom).

The SOP experiment set out to discover what relationships could be established between decisions made during the production of bourbon whiskey and the sensory character of the resulting product. The model, predicated on the use of THD as an encoding of manifold structure, is able to offer an interpretable setting for performing feature analysis and object modeling. Further, such a model may enable leverage chemical composition as a shared basis for understanding variances and relationships within and between multiple modalities of metadata in experiments involving the analysis of complex mixtures. As discussed herein, some embodiments establish a methodology capable of identifying these relationships by way of an empirical linkage to the product's chemical composition. The automated discovery of whiskey chemical and flavor profiles, together with their associations with production parameters, make room for unprecedented scientific and practical understandings of the intricacies underlying the production of whiskey. Moreover, our technique is general enough to extend to analyses of other distilled spirits, of experiments relying on chemometrics, and of multimodal datasets broadly.

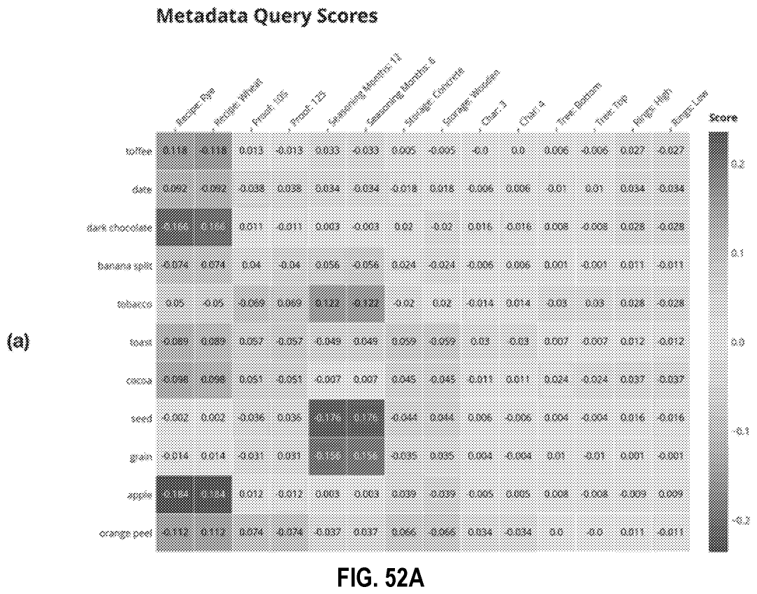

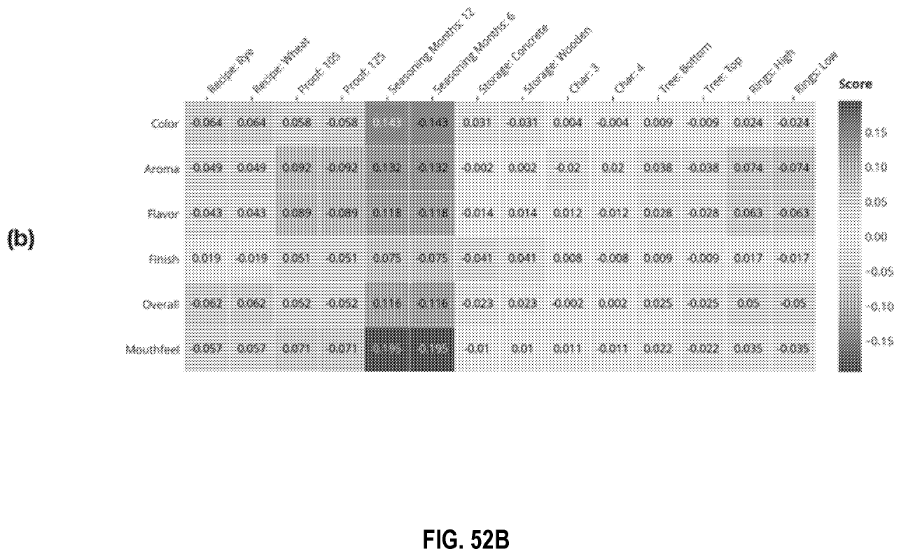

The analysis revealed numerous relationships between sensory characteristics, consumer preferences, and production variables underlying the results of the SOP experiment. FIGS. 52A and B show some of the results obtained from the example calculation of query matrices for production and sensory metadata as well as production and consumer preference metadata. In aggregate, consumers exhibited the strongest preference for barrels whose staves were seasoned for twelve months. Less pronounced yet still detectable were preferences for barrels which were filled at 105 proof, for barrels whose mash was flavored with wheat grains, and a slight preference for tighter stave gain size. This order of preference contrasts with the averages derived from the raw consumer preference scores alone, which have been analyzed previously. Based on the average ratings alone, consumers exhibited the strongest preference for barrels aged in wooden rickhouses. This was followed by preferences for twelve months of barrel stave seasoning, a mash flavored with wheat, and a barrel entry proof of 105.

FIGS. 52A and B depict query results for experimental metadata in this example. Query scores summarize the total co-enrichment strength between metadata features across the full set of local feature clusters. FIG. 52A depicts query scores calculated between a sample of the sensory metadata features and the full set of production metadata features. FIG. 52B depicts query scores calculated between the customer ratings metadata features and production metadata features.

Sensory data may be received from any number of sources. In this example, sensory data may be scraped from the reviews of the full set of Single Oak Project barrels on the website Drinkhacker. Sensory descriptors may be identified and related manually in a hierarchical flavor ontology. A binary vector representation may be created for each barrel by enumerating each sensory token in the ontology and assigning a value of 1 to tokens which either appear in the review or whose children in the ontology appear in the review, and 0 otherwise. Customer ratings (e.g., collected by Buffalo Trace Distillery) may be transformed by calculating a Wilson confidence interval lower bound at a confidence level of 0.9 on a beta distribution modeling the likelihood of a barrel receiving an above-average rating.

A more detailed analysis utilizing THD clustering reveals several sets of barrels which consumers exhibit strong preferences for despite being composed of one or more production variables which were not individually preferred on average. The highest-rated clusters and their corresponding production metadata enrichment scores. Additionally, performing THD clustering of the data matrix of production and sensory query scores reveals several configurations of production variables which yield distinct sensory profiles.

It will be appreciated that the application as discussed herein is one of many examples. The use of chemical data and the use of query scores in combination with THD analysis reveal more complex and intricate configurations of production variables that may be more strongly preferred than those indicated by broad correlation. Additionally, the formation of specific sensory or profiles (e.g., chemical, flavor, or the like) as a result of choices in production variables is detectable using similar analytical methods.

Further, it will be appreciated that some embodiments will be valuable to simultaneously integrate features across a variety of chemical collection methods. While GC-FID is well suited for capturing volatile compounds which play an essential role in sensory perception of whiskey products, methods such as nuclear magnetic resonance (NMR) or liquid chromatography with mass spectrometry (LC-MS) may be sensitive to more complex organic compounds such as those extracted from the wooden barrel staves during maturation. This difference in sensitivity may lead to a more comprehensive understanding of sensory perception of whiskey and may help to elucidate the specific impact that decisions governing barrel production may have.

Moreover, different embodiments may be utilized for any chemical process. It will be appreciated that systems and methods that apply to bourbon or whiskey may be applied to any other libation, including, but not limited to, wine and/or spirits. It will be apparent that utilizing systems and methods described herein may enable creation of a platform that broadens wine and spirits market reach and enhances consumer experiences. This may include generating data-driven insights that link chemical composition to customer preferences and improved customer experiences.

Similar to bourbon and whiskey as described herein, human taste and perception vary greatly among individuals and across populations. Consumers primarily rely on the following sources for insights: internet reviews, word-of-mouth and community feedback, marketing and advertising campaigns, and decisions based on the “other people's opinions” for purchases.

No current capability exists that can: accurately maps chemical signatures to customer preferences, forecast the market reception of upcoming products, or take the subjectiveness out of customer preferences.

Using systems and methods described herein to wine and/or spirits, some embodiments may link personal tastes to the inherent chemical makeup of wines or spirits, lead to accurate matching of individual and groups to specific preferences, combine tasting notes and reviews to

derive insights from broad to individual sensory preferences, and generation of applications designed to suggest brands to users based on entered descriptions (e.g., personalized analytics).

As a result, recommendations tailored to individual sensory preferences may be generated, thereby moving away from the less relevant “frequently purchased together” data, and enable users to submit bourbon reviews and compare with feedback from others on similar products. As a result, multiple revenue streams may include: improved ad revenue, possible subscription fees for application, and/or exclusive partnerships with distillers and vineyards for product optimization.

Exemplary embodiments are described herein in detail with reference to the accompanying drawings. However, the present disclosure can be implemented in various manners, and thus should not be construed to be limited to the embodiments disclosed herein. On the contrary, those embodiments are provided for the thorough and complete understanding of the present disclosure, and completely conveying the scope of the present disclosure.

It will be appreciated that aspects of one or more embodiments may be embodied as a system, method, or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer-readable medium(s) having computer-readable program code embodied thereon.

Any combination of one or more computer-readable medium(s) may be utilized. The computer-readable medium may be a computer-readable signal medium or a computer-readable storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), a solid state drive (SSD), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program or data for use by or in connection with an instruction execution system, apparatus, or device.

A transitory computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof.

Program code embodied on a computer-readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++, Python, or the like and conventional procedural programming languages, such as the “C” programming language or similar programming languages. The computer program code may execute entirely on any of the systems described herein or on any combination of the systems described herein.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer, other programmable data processing apparatus, or other devices to function in a particular manner, such that the instructions stored in the computer-readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

While specific examples are described above for illustrative purposes, various equivalent modifications are possible. For example, while processes or blocks are presented in a given order, alternative implementations may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed or implemented concurrently or in parallel or may be performed at different times. Further any specific numbers noted herein are only examples: alternative implementations may employ differing values or ranges.

Throughout this specification, plural instances may implement components, operations, or structures described as a single instance. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. Furthermore, any specific numbers noted herein are only examples: alternative implementations may employ differing values or ranges.

Components may be described or illustrated as contained within or connected with other components. Such descriptions or illustrations are examples only, and other configurations may achieve the same or similar functionality. Components may be described or illustrated as “coupled”, “couplable”, “operably coupled”, “communicably coupled” and the like to other components. Such description or illustration should be understood as indicating that such components may cooperate or interact with each other, and may be in direct or indirect physical, electrical, or communicative contact with each other.

Components may be described or illustrated as “configured to”, “adapted to”, “operative to”, “configurable to”, “adaptable to”, “operable to” and the like. Such description or illustration should be understood to encompass components both in an active state and in an inactive or standby state unless required otherwise by context.

It may be apparent that various modifications may be made, and other embodiments may be used without departing from the broader scope of the discussion herein. Therefore, these and other variations upon the example embodiments are intended to be covered by the disclosure herein

1. A non-transitory computer readable medium comprising executable instructions, the executable instructions being executable by one or more processors to perform a method, the method comprising:
receiving multimodal analysis data including chemical data and sensory data;
generating an n-dimensional feature space representation of the multimodal analysis data;
projecting the feature space into a manifold embedding using a similarity metric;
performing recursive hierarchical decomposition of the manifold embedding using non-overlapping coverings to generate a connected-component network including branch points;
performing a local transform operation for leaf nodes of the connected-component network to generate local object embeddings;
computing co-enrichment scores between features of different modalities based on co-localization within the local object embeddings;
generating a unified multimodal representation based on the co-enrichment scores; and
generating a visualization of the unified multimodal representation, the visualization including nodes and relationships representing correlations between the modalities.

2. The non-transitory computer-readable medium of claim 1, wherein the chemical data comprises gas chromatography data.

3. The non-transitory computer-readable medium of claim 1, wherein preprocessing includes baseline correction and peak alignment.

4. The non-transitory computer-readable medium of claim 1, wherein the embedding uses a nonlinear dimensionality reduction technique.

5. The non-transitory computer-readable medium of claim 1, wherein recursive hierarchical decomposition includes generating coverings at multiple resolutions.

6. The non-transitory computer-readable medium of claim 1, wherein co-enrichment scores quantify co-localization of features.

7. The non-transitory computer-readable medium of claim 1, wherein multimodal fusion includes merging connected-component networks.

8. The non-transitory computer-readable medium of claim 1, wherein peak alignment includes time-warping.

9. The non-transitory computer-readable medium of claim 1, wherein co-enrichment is computed using similarity distributions.

10. The non-transitory computer-readable medium of claim 1, wherein embeddings preserve similarity relationships.

11. A system comprising at least one processor and memory containing instructions, the instructions being executable by the at least one processor to:
receiving multimodal analysis data including chemical data and sensory data;
generate an n-dimensional feature space representation of the multimodal analysis data;
project the feature space into a manifold embedding using a similarity metric;
perform recursive hierarchical decomposition of the manifold embedding using non-overlapping coverings to generate a connected-component network including branch points;
perform a local transform operation for leaf nodes of the connected-component network to generate local object embeddings;
compute co-enrichment scores between features of different modalities based on co-localization within the local object embeddings;
generate a unified multimodal representation based on the co-enrichment scores; and
generate a visualization of the unified multimodal representation, the visualization including nodes and relationships representing correlations between the modalities.

12. The system of claim 11, wherein the chemical data comprises gas chromatography data.

13. The system of claim 11, wherein preprocessing includes baseline correction and peak alignment.

14. The system of claim 11, wherein the embedding uses a nonlinear dimensionality reduction technique.

15. The system of claim 11, wherein recursive hierarchical decomposition includes generating coverings at multiple resolutions.

16. The system of claim 11, wherein co-enrichment scores quantify co-localization of features.

17. The system of claim 11, wherein multimodal fusion includes merging connected-component networks.

18. The system of claim 11, wherein peak alignment includes time-warping.

19. The system of claim 11, wherein co-enrichment is computed using similarity distributions.

20. The system of claim 11, wherein embeddings preserve similarity relationships.

21. A method comprising:
receiving multimodal analysis data including chemical data and sensory data;
generating an n-dimensional feature space representation of the multimodal analysis data;
projecting the feature space into a manifold embedding using a similarity metric;
performing recursive hierarchical decomposition of the manifold embedding using non-overlapping coverings to generate a connected-component network including branch points;
performing a local transform operation for leaf nodes of the connected-component network to generate local object embeddings;
computing co-enrichment scores between features of different modalities based on co-localization within the local object embeddings;
generating a unified multimodal representation based on the co-enrichment scores; and
generating a visualization of the unified multimodal representation, the visualization including nodes and relationships representing correlations between the modalities.
